(19) United States
(12) Patent Application Publication
(10) Pub. No.: US 2004/0224981 A1
(43) Pub. Date: Nov. 11, 2004

(54) Antibacterial methods and compositions

(76) Inventors: Nebojsa Janjic, Boulder, CO (US); Ian A. Critchley, Lafayette, CO (US); Joseph Guiles, Lafayette, CO (US); Theodore M. Tarasow, Longmont, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/837,881

(22) Filed: May 3, 2004

Related U.S. Application Data
(60) Provisional application No. 60/467,377, filed on May 1, 2003.
(60) Provisional application No. 60/486,482, filed on Jul. 10, 2003.

(51) Int. Cl. A61K 31/47; A61K 31/4709

(52) U.S. Cl. 514/312; 514/394; 514/313

(57) ABSTRACT

Disclosed is a pharmaceutical composition comprising an aminoacyl tRNA synthetase inhibitor and another antibacterial agent, including another aminoacyl tRNA synthetase inhibitor.

### CROSS-REFERENCE TO RELATED APPLICATIONS

This application claims the benefit of U.S. Provisional Application No. 60/467,377, filed May 1, 2003, and also claims the benefit of U.S. Provisional Application No. 60/486,482, filed Jul. 10, 2003, each entitled “Antibacterial Methods And Compositions.” Each of these applications is incorporated by reference herein in their entirety.

### BACKGROUND OF THE INVENTION

Antibacterials kill or inhibit the growth of bacteria by interfering with major processes of cellular function that are essential for survival. The β-lactams (penicillins and cephalosporins) and the glycopeptides (vancomycin and teicoplanin) inhibit synthesis of the cell wall. Macrolides (erythromycin, clarithromycin, and azithromycin), clindamycin, chloramphenicol, aminoglycosides (streptomycin, gentamicin, and amikacin) and the tetracyclines inhibit protein synthesis. Also inhibiting protein synthesis is the newest class of antibacterials to be approved (linezolid) are synthetic oxazolidinones. Rifampin inhibits RNA synthesis, the fluoroquinolones (such as ciprofloxacin) inhibit DNA synthesis indirectly by inhibiting the enzymes that maintain the topological state of DNA. Trimethoprim and the sulfonamides inhibit folate biosynthesis directly and DNA synthesis indirectly by depleting the pools of one of the required nucleotides (Chambers, H. F. and Sande, M. A. (1996) Antimicrobial Agents. Goodman & Gilman's The Pharmacological Basis of Therapeutics, McGraw-Hill, New York). The disclosure of this reference, and of all other patents, patent applications, and publications referred to herein, are incorporated by reference herein in their entirety.

Resistance to antibacterials can occur when the target of a drug mutates so that it can still function, but is no longer inhibited by the drug (e.g., mutations in the quinolone resistance determining regions of bacterial gyrases and topisomerase enzymes that confer resistance to the fluoroquiniolones). Resistance may also be mediated by the over-expression or activation of efflux pumps that remove the drug from the cell interior (e.g. tetracycline efflux). Another common mechanism of resistance involves the production of enzymes that modify or degrade the drug so that it becomes inactive (e.g., β-lactamases, aminoglycoside modifying enzymes, etc.). Because of the growth advantage this gives to the resistant cells and their progeny in the presence of the antibacterial, the resistant organisms quickly take over a population of bacteria. Resistance developed in one cell can be transferred to other bacteria in the population since bacteria have mechanisms for directly exchanging genetic material. In a recent congressional report, the General Accounting Office (GAO) has summarized the current and future public health burden resulting from drug-resistant bacteria (Antimicrobial Resistance (1999). General Accounting Office (GAO/RCED-99-132)). According to this report, the number of patients treated in a hospital setting for an infection with drug-resistant bacteria has doubled from 1994 to 1996 and again almost doubled from 1996 to 1997. Resistant strains can spread easily in environments such as hospitals or tertiary care facilities that have a sizeable population of immunosuppressed patients. The same GAO report also provides clear evidence that previously susceptible bacteria are increasingly becoming resistant and spreading around the world. Furthermore, the proportion of resistant bacteria within bacterial populations is on the rise. An especially frightening development is the appearance of bacterial strains that are multi-resistant or even pan-resistant to all approved antibacterials. Recognizing the dramatic increase of drug-resistant bacteria, the Food and Drug Administration has recently issued a recommendation urging physicians to use antibacterials more judiciously and only when clinically necessary (FDA Advisory (2000). Federal Register 65 (182), 56511-56518).

These circumstances have prompted efforts to develop new antibiotics that overcome the emerging antibiotic-resistant bacteria. The aminoacyl tRNA synthetases are essential enzymes found in all living organisms. These enzymes have emerged as attractive targets for the development of new antibiotics, since compounds that inhibit these enzymes have the ability to circumvent existing resistance mechanisms.

Pseudomonic acid A, also known as mupirocin, is a natural product synthesized by Pseudomonas fluorescens and is an inhibitor of isoleucyl-tRNA synthetases from Gram-positive infectious pathogens, including S. aureus, S. epidermidis, and S. saprophyticus and Gram-negative organisms, such as Haemophilus influenzae, Neisseria gonorrhoeae, and Neisseria meningitides. The bacterial isoleucyl tRNA synthetase enzyme has been successfully targeted by mupirocin or a pharmaceutically acceptable salt when formulated as an ointment or cream for the topical therapy of bacterial skin infections. Mupirocin and derivatives are mainly active against Gram-positive aerobes and some Gram-negative aerobes. Mupirocin free acid, its salts and esters are described in UK patent No. 1,395,907. These agents are found to be useful in treating skin, ear and eye disorders.

Three commercial products contain mupirocin free acid or crystalline mupirocin calcium dihydrate as the active ingredients. These products are Bactroban® Ointment, Bactrobang Nasal and Bactroban® Cream, manufactured by GlaxoSmithKline. The first contains mupirocin, while the other two contain crystalline mupirocin calcium dihydrate. The formulation of Bactrobang Ointment is described in U.S. Pat. No. 4,524,075. The formulation of Bactrobang Nasal is described in U.S. Pat. No. 4,790,989. The cream base of Bactrobang Cream is described in WO 95/10999 and U.S. Pat. No. 6,025,389.

Crystalline mupirocin calcium, its properties and methods of preparation are described in detail in U.S. Pat. No. 4,916,155. This patent emphasizes the improved thermal stability of the crystalline dihydrate form of the calcium salt. Mupirocin calcium amorphous has been described in U.S. Pat. No. 6,489,358.

Although mupirocin is a widely accepted and successful product, two types of resistance have been described: 1) Low level resistance with minimum inhibitory concentrations (MIC's) in the range of 8-256 μg/mL that is largely attributed to mutations in the chromosomally encoded isoleucyl tRNA synthetase protein, and 2) High level resistance (mupA) that is caused by a plasmid-encoded IRS enzyme and results in MICs>512 μg/mL.

In addition, a recent surveillance study conducted in 2000 has identified mupirocin resistance rates in oxacillin-resistant Staphylococcus aureus ranging from 4.6% in Latin America to 14.1% and 17.8% in North America and Europe respectively.

Other known natural product inhibitors directed against aminoacyl tRNA synthetases included borrelidin, furanomycin, granaticin, indolmycin, ochartoxin A, and cispentacin, although none has been developed into an antibiotic to date. Methionyl tRNA sythetase inhibitors include 2-NH-pyridones and pyrimidone methionyl t-RNA synthetase inhibitors as described in International Patent Application Publication WO 00/71524; benzimidazole derivatives which are methionyl t-RNA synthetase inhibitors as described in International Patent Application Publication WO 00/71522; methionyl t-RNA synthetase inhibitors as described in International Patent Application Publication WO 99/55677 and WO 00/21949; 2-(NH—or O—substituted) quinolones which are inhibitors of methionyl t-RNA synthetase as described in U.S. Pat. No. 6,320,051; U.S. patent application Ser. No. 10/729,416, filed Dec. 5, 2003, entitled “2-NH-Heteroarylimidazoles with Antibacterial Activity;” International Patent Application Ser. No. PCT/US2004/03040, filed Feb. 2, 2004, entitled “Novel Compounds;” Jarvest, et al., Bioorganic & Medicinal Chemistry Letters (2003) 13:665-668; Jarvest, et al., (2002) J. Med. Chem. 45: 1959-1962; and U.S. patent application Ser. No. 10/789,811, filed Feb. 27, 2004, entitled “Substituted Thiophenes with Antibacterial Activity.” There remains a need for formulations of tRNA synthetase inhibitors as antibacterial agents.

### SUMMARY OF THE INVENTION

The present invention provides a pharmaceutical composition comprising an aminoacyl tRNA synthetase inhibitor and another antibacterial agent, including another aminoacyl tRNA synthetase inhibitor.

### BRIEF DESCRIPTION OF THE FIGURES

FIG. 1 shows selection of spontaneous resistant mutants from S. aureus ATCC 29213 following exposure to MRSi compound 2 and mupirocin alone and in combination.

FIG. 2 shows selection of spontaneous resistant mutants from S. aureus 31-1334 (low level mupirocin-resistant) following exposure to MRSi compound 2 and mupirocin alone and in combination.

FIG. 3 shows selection of spontaneous resistant mutants from seven staphylococcal isolates following exposure to MRSi compound 5 alone, mupirocin alone and MRSi compound 5/mupirocin combination.

FIG. 4 shows growth curves for low-level MRS-resistant strains of S. aureus (SP-1A2 and SP-1B5) and their wild type MRS-susceptible parent strain.

### DETAILED DESCRIPTION OF THE INVENTION

One embodiment of the present invention is a therapeutic composition that, when administered to a host in an effective manner, is capable of protecting that human or animal from disease caused by bacteria. As used herein, a protective compound refers to a compound that, when administered to a human or animal in an effective manner, is able to treat, ameliorate, and/or prevent disease caused by bacteria.

The present disclosure describes therapeutic combination compositions containing at least one aminoacyl tRNA synthetase inhibitor, particularly a methionyl tRNA synthetase inhibitor, for use as antibacterial agents. The benefits of such combination therapy are not limited to topical uses but extend to oral and parenteral administration. The therapeutic compositions are useful for the prevention and/or treatment of infections caused by organisms that are resistant to mupirocin and other currently marketed antimicrobial agents.

In one embodiment, the invention contemplates formulations comprising at least one aminoacyl tRNA synthetase inhibitor in combination with at least one additional therapeutic agent, preferably an antibacterial or antibiotic agent, as active ingredients for the therapy of bacterial infections.

In one embodiment, the therapeutic composition contains a methionyl aminoacyl tRNA synthetase (MRS) inhibitor. MRS inhibitors are described in International Patent Application Publications WO 00/71524, WO 00/71522, WO 99/55677 and WO 00/21949; U.S. Pat. No. 6,320,051; U.S. patent application Ser. No. 10/729,416, filed Dec. 5, 2003, entitled “2-NH-Heteroarylimidazoles with Antibacterial Activity;” International Patent Application Ser. No. PCT/US2004/03040, filed Feb. 2, 2004, entitled “Novel Compounds;” Jarvest, et al., Bioorganic & Medicinal Chemistry Letters (2003) 13:665-668; Jarvest, et al., (2002) J. Med. Chem. 45: 1959-1962; and U.S. patent application Ser. No. 10/789,811, filed Feb. 27, 2004, entitled “Substituted Thiophenes with Antibacterial Activity,” and are exemplified herein by the following compounds:

The MRS inhibitors,

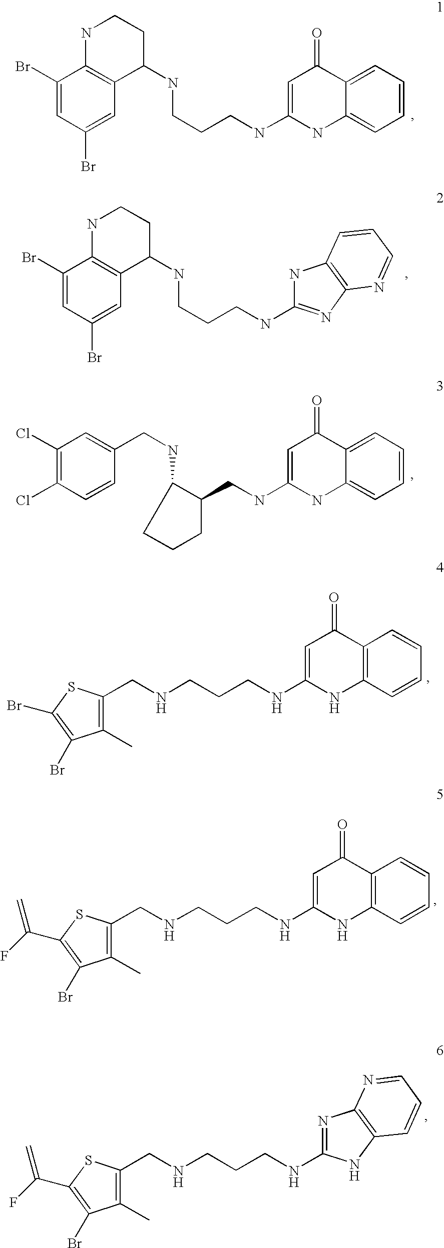

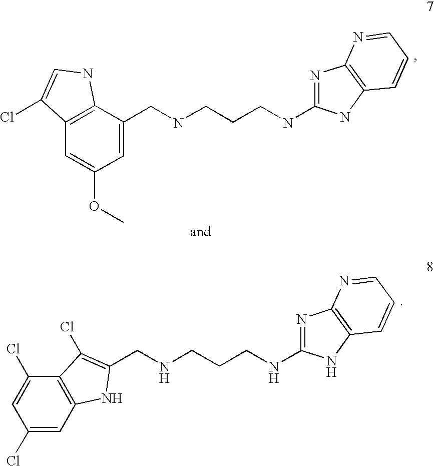

The MRS inhibitors 1-8 may also be referred to, respectively, as 2-[3-(6,8-Dibromo-2,3,4,5-tetrahydroquinolin-4-ylamino)prop-1-ylamino]-1H-quinolin-4-one; N-(6,8-Dibromo-1,2,3,4-tetrahydroquinolin-4-yl)-N′-(1H-imidazo[4,5-b]pyridine-2-yl)-propane-1,3-diamine dihydrochloride; 2-{[(1R, 2S)-2-(3,4-Dichlorobenzylamino)cyclopentylmethyl]amino}-1H-quinolin-4-one; N-(4,5-Dibromo-3-methylthiophen-2-ylmethyl)-N′-(1H-quinolin-4-one)propane-1,3-diamine; N-(4-bromo-5-(1-fluorovinyl)-3-methylthiophen-2-ylmethyl)-N′-(1H-quinolin-4-one)propane-1,3-diamine; N-(4-bromo-5-(1-fluorovinyl)-3-methylthiophen-2-ylmethyl)-N′-(1H-imidazo[4,5-b]pyridin-2-yl)-propane-1,3-diamine; N-(3-Chloro-5-methoxy-1H-indol-7-ylmethyl)-N′-(1H-imidazo[4,5-b]pyridin-2-yl)-propane-1,3-diamine; and N-(1H-imidazo[4,5-b]pyridin-2-yl)-N′-(3,4,6-trichloro-1H-indol-2-ylmethyl)—propane-1,3-diamine.

In one embodiment, the therapeutic composition contains an MRS inhibitor and Mupirocin or Fusidic Acid. Mupirocin or Fusidic Acid may be used in their pharmaceutically acceptable salt or ester. In addition the therapeutic composition may be in the form of MRS inhibitor mupirocinate (i.e. salt formed between MRS inhibitor and Mupirocin) or MRS inhibitor Fusidate (i.e. salt formed between MRS inhibitor and Fusidic acid). MRS inhibitor may be interchangeably referred to as MRSi for brevity. Suitable pharmaceutically acceptable salts of Mupirocin or Fusidic Acid are well known in the art and include alkali metal salts such as sodium and lithium and alkaline earth metal salts such as calcium, of which the calcium salt is desirable, in particular the crystalline dihydrate form thereof, as well as other metal salts, for instance silver and aluminium salts and ammonium substituted ammonium salts. The salts may be anhydrous or may be in the form of pharmaceutically acceptable solvates, for instance alcoholates and, especially, hydrates. Salts can include the calcium, silver and lithium salts, in particular the calcium salt. In the case of the calcium salt of mupirocin, the crystalline salt, the crystalline hydrated calcium salt, or the crystalline dihydrate salt, is used. The MRSi mupirocinate salt or the MRSi Fusidate may be in the form of pharmaceutically acceptable solvates, for instance alcoholates and, especially, hydrates.

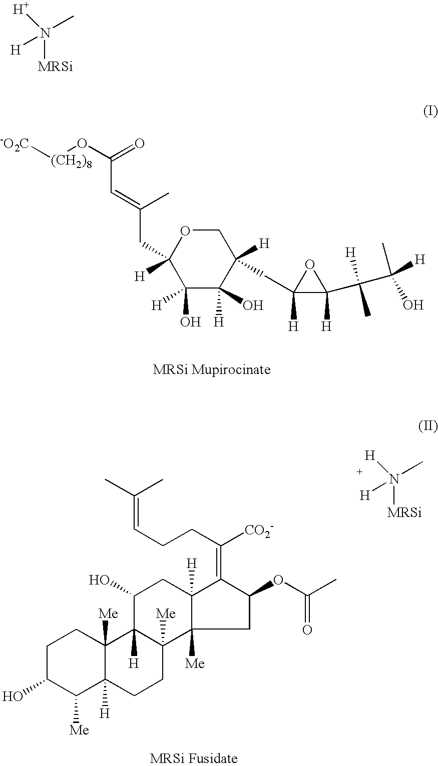

In one embodiment the bacterial infections are topical bacterial infections including but not limited to impetigo, infected skin lesions, infected dermatitis (eczema, psoriasis, etc.), wound infections, burn infections, post-operative infections, dialysis site infections, and infections associated with colonization of the nasopharyrx by pathogenic organisms, sinusitis, including recurrences.

Where the active ingredients of the therapeutic composition are aminoacyl tRNA synthetase inhibitors, two or more inhibitors in combination in a therapeutic composition of the invention should show synergy or additivity since each inhibitor targets a component of the same biochemical process (charging of tRNAs with cognate amino acids or, more generally, protein synthesis). In addition, an antibacterial drug comprised of a combination of tRNA synthetase inhibitors would have a low propensity for the development of resistance since resistance in two enzymes would need to develop simultaneously to confer protection of bacteria against the drug. A combined product embodied in this invention will have the ability to circumvent both the low- and high-level mupirocin (mupA) resistance mechanisms that have arisen in clinical isolates. Such as combined product would also not suffer from the disadvantage of exposing the bacteria to low level dosages of drug substances that might increase the risk of the development of resistant bacteria in a formulation with a single drug substance. Although many of the amino acyl tRNA synthetase inhibitors described to date are bacteriostatic, the combined product would be expected to show bactericidal activity at local concentrations at the site of infection since high doses can be applied topically.

Any tRNA synthetase inhibitors known in the art can be used in combination in accordance with this disclosure. In addition to those described above, tRNA synthetase inhibitors useful in the present invention include but are not limited to borredidin, furanomycin, granaticin, indolmycin, ochratoxin A, cispentacin, 5′-O-glycylsulfamoyladenosine; proline-based t-RNA synthetase inhibitors described in U.S. Patent Application Publication 2003-0013724A1, and U.S. Pat. Nos. 6,417,217 and 6,333,344; aminoacyl sulfamide-based t-RNA synthetase inhibitors described in U.S. Pat. No. 5,824,657; catechol-based t-RNA synthetase inhibitors as described in U.S. Pat. No. 6,348,482 and U.S. Patent Application Publication 2002-0040147A1; heterocycle-based tRNA synthetase inhibitors as described in U.S. Pat. No. 6,153,645; aminoacyl adenylate mimic isoleucyl-tRNA synthetase inhibitors as described in U.S. Pat. No. 5,726,195; oxazolone derivatives as described in U.S. Pat. Nos. 6,414,003 and 6,169,102; and oligonucleotides targeted to a region of the cloverleaf structure of a tRNA as described in U.S. Pat. No. 6,448,059.

The therapeutic compositions of the present disclosure have antibacterial activity against clinically important Gram-positive pathogens including the staphylococci, streptococci and enterococci and particularly including isolates resistant to currently marketed agents.

The therapeutic compositions of the present disclosure can also be used for the prevention and/or treatment of infections caused by organisms that are resistant to mupirocin and other currently marketed antimicrobial agents.

For topical application to the skin or mucus membranes of the nose and throat, including the nasopharynx, the active ingredient(s) may be made up into a cream, lotion ointment, sprays or inhalants, lozenges, throat paints, dentifrices, powders, encapsulated in micelles or liposomes and drug release capsules including the active compounds incorporated within a biocompatible coating designed for slow-release, and mouthwashes and other washes. Formulations which may be used for the active ingredient are conventional formulations well known in the art, for example as described in standard textbooks of pharmaceutics such as the United States Pharmacopoeia (USP), British Pharmacopoeia, European Pharmacopoeia, Japanese Pharmacopoeia, and International Pharmacopoeia.

The compositions of the present disclosure may be made up in any conventional carriers suitable for the topical administration of antibiotics, for example paraffins and alcohols. They may be presented, as, for instance, ointments, creams or lotions, eye and ear ointments, gels, skin patches, impregnated dressings and aerosols. The compositions may also contain appropriate conventional additives, for example preservatives, solvents to assist drug penetration (e.g., DMSO), emollients, local anesthetics, preservatives and buffering agents.

A suitable composition according to the present invention comprises about 0.01% to 99% by weight, preferably 0.1-40% by weight, of the active ingredient. If the compositions contain dosage units, each dosage unit preferably contains from 0.1-500 mg of the active material. For adult human treatment, the dosage employed preferably ranges from 1 mg to 5 g, per day, depending on the route and frequency of administration of each of the tRNA synthetase inhibitors.

A suitable ointment base may conveniently comprise from 65 to 100% (preferably 75 to 96%) of white soft paraffin, from 0 to 15% of liquid paraffin, and from 0 to 7% (preferably 3 to 7%) of lanolin or a derivative of synthetic equivalent thereof. Another suitable ointment base may conveniently comprise a polyethylene—liquid paraffin matrix.

A suitable cream base may conveniently comprise an emulsifying system, for example from 2 to 10% of polyoxyethylene alcohols (e.g. the mixture available under the trade mark Cetomacrogol 1000), from 10 to 25% of stearyl alcohol, from 20 to 60% of liquid paraffin, and from 10 to 65% of water; together with one or more preservatives, for example from 0.1 to 1% of N,N″-methylenebis[N′-[3-(hydroxymethyl)-2,5-dioxo-4-imidazolidinyl]urea] (available under the name Imidurea USNF), from 0.1 to 1% of alkyl 4-hydroxybenzoates (for example the mixture available from Nipa Laboratories under the trade mark NIPASTAT), from 0.01 to 0.1% of sodium butyl 4-hydroxybenzoate (available from Nipa Laboratories under the trade mark NIPABUTYL SODIUM), and from 0.1 to 2% of phenoxyethanol.

Other suitable bases for creams include sorbitan monostearate, Polysorbate 60, cetyl palmitate, paraffin, cetylstearyl alcohol, benzyl alcohol, silica, triacetin, isopropyl monostearate, polyethylene glycol, glycerol monostearate, polyacrylic acid, sodium hydroxide, docusate sodium, dimethicone, triglycerides, octyldecanol and octyldodecanol. In some embodiments, there is provided a cream preparation which comprises an oleaginous base selected from the group consisting of petrolatum and hard fat; stiffening agents that are selected from the group consisting of cetostearyl alcohol, cetyl alcohol and stearyl alcohol; humectants selected from a group consisting of castor oil and oleyl alcohol; surfactants selected from the group consisting of a surfactant with an HLB equal to or below 5, and other pharmaceutically accepted additives.

A suitable gel base may conveniently comprise a semi-solid system in which a liquid phase is constrained within a three dimensional polymeric matrix with a high degree of cross-linking. The liquid phase may conveniently comprise water, together with from 0 to 20% of water-miscible additives, for example glycerol, polyethylene glycol, or propylene glycol, and from 0.1 to 10%, preferably from 0.5 to 2%, of a thickening agent, which may be a natural product, for example tragacanth, pectin, carrageen, agar and alginic acid, or a synthetic or semi-synthetic compound, for example methylcellulose and carboxypolymethylene (carbopol); together with one or more preservatives, for example from 0.1 to 2% of methyl 4-hydroxybenzoate (methyl paraben) or phenoxyethanol. Another suitable base may comprise from 70 to 90% of polyethylene glycol (for example, polyethylene glycol ointment containing 40% of polyethylene glycol 3350 and 60% of polyethylene glycol 400, prepared in accordance with the U.S. National Formulary (USNF)), from 5 to 20% of water, from 0.02 to 0.25% of an anti-oxidant (for example butylated hydroxytoluene), and from 0.005 to 0.1% of a chelating agent (for example ethylenediamine tetraacetic acid (EDTA)).

The term soft paraffin as used above encompasses the cream or ointment bases white soft paraffin and yellow soft paraffin. The term lanolin encompasses native wool fat and purified wool fat. Derivatives of lanolin include in particular lanolins which have been chemically modified in order to alter their physical or chemical properties and synthetic equivalents of lanolin include in particular synthetic or semisynthetic compounds and mixtures which are known and used in the pharmaceutical and cosmetic arts as alternatives to lanolin and may, for example, be referred to as lanolin substitutes.

One suitable synthetic equivalent of lanolin that may be used is the material available under the SOFTISAN trade mark.

The compositions of the disclosure may be produced by conventional pharmaceutical techniques. Thus the aforementioned composition, for example, may conveniently be prepared by mixing together at an elevated temperature, preferably 60-70° C., the soft paraffin, liquid paraffin if present, and lanolin or derivative or synthetic equivalent thereof. The mixture may then be cooled to room temperature, and, after addition of active ingredients and any other ingredients, stirred to ensure adequate dispersion. If necessary the composition may be milled at any suitable stage of the process. A suitable sterilization procedure may also be included if necessary. Alternatively raw materials are obtained in sterile condition and the compositions are produced aseptically.

Generally, the therapeutic agents used in the disclosure are administered to a human or animal in an effective amount. Generally, an effective amount is an amount effective to either (1) reduce the symptoms of the disease sought to be treated or (2) induce a pharmacological change relevant to treating the disease sought to be treated. For bacterial infections, an effective amount includes an amount effective to: reduce or eliminate the bacterial population; slow the spread of infection; or increase the life expectancy of the affected human or animal.

Therapeutically effective amounts of the therapeutic agents can be any amount or doses sufficient to bring about the desired effect and depend, in part, on the condition, type and location of the infection, the size and condition of the patient, as well as other factors readily known to those skilled in the art. The dosages can be given as a single dose, or as several doses, for example, divided over the course of several weeks.

The present disclosure is also directed toward methods of treatment utilizing the therapeutic compositions of the present disclosure. The method comprises administering the therapeutic agent to a subject in need of such administration.

Compositions may be applied topically both to the outer skin and to other parts of the human or animal body, for example the eyes and inside the nose. The compositions may also be applied topically to areas in which the skin is missing or damaged, as found, for example, in burns and wounds.

Thus, the present disclosure provides a method of treating skin disorders in human or domestic mammals, which method comprises applying topically to a human or domestic mammal in need thereof the composition.

In addition, the present invention, in some embodiments, also provides for the use of a tRNA synthetase inhibitor with another antibiotic including another tRNA synthetase inhibitor such as mupirocin or a pharmaceutically acceptable ester or salt thereof in the manufacture of a medicament for the prophylactic treatment of infections including, but not limited to, surgical site infections, catheter-associated infections, burns and sinusitis, including recurrent infections.

Such treatment may be prophylactic treatment; that is treatment that includes not only complete elimination of the bacterial infection, but also a partial elimination of thereof, that is a reduction in the number of acute episodes.

It is believed that the successful treatment of bacterial infections, such as recurrent otitis media and recurrent sinusitis, is associated with the elimination or reduction of nasal carriage of pathogenic bacteria such as S. aureus, H. influenzae, S. pneumoniae and M. catarrhalis, in particular colonization of the nasospharynx by such organisms.

Accordingly, in a further aspect, the present invention provides for the use of tRNA synthetase inhibitor with another antibiotic including another tRNA synthetase inhibitor such as mupirocin or a pharmaceutically acceptable ester or salt thereof in the manufacture of a medicament for reducing or eliminating the nasal carriage of pathogenic organisms associated with recurrent otitis media, which medicament is adapted for nasal administration, in particular, focused delivery to the nasopharynx.

### EXAMPLES

### EXAMPLE 1

### General Method for Mupirocinate Salt Formation

To 1.0 meq of a methanolic Psuedomonic acid A solution was added 1.0 meq of an MRSi. The mixture is warmed to 50° C. and gently agitated for 10 minutes. After the mixture returns to ambient temperature it is filtered through a 1 μm glass fiber syringe filter. The flask and filter are rinsed with methanol and the combined filtrates diluted with water. The solution was then concentrated using a centrifugal concentrator followed by drying in a vacuum oven at ambient temperature to give an off-white amorphous solid.

### EXAMPLE 2

### General Method for Fusidate Formation

To 1.0 meq of a methanolic Fusidic acid solution was added 1.0 meq of an MRSi. The mixture is warmed to 50° C. and gently agitated for 10 minutes. After the mixture returns to ambient temperature it is filtered through a 1 μm glass fiber syringe filter. The flask and filter are rinsed with methanol and the combined filtrates diluted with water. The solution was then concentrated using a centrifugal concentrator followed by drying in a vacuum oven at ambient temperature to give an off-white amorphous solid.

### EXAMPLE 3

### 2-13-(6,8-DIBROMO-2,3,4,5-TETRAHYDROQUINOLIN-4-YLAMINO)PROP-1-YLAMINOI-1H-QUINOLIN-4-ONE.

A solution of 2-(3-aminopropylamino)-1H-quinolin-4-one dihydrochloride (0.038 g, 0.13 mmol) in methanol (2 ml) and acetic acid (0.1 ml) was treated with sodium methoxide (0.5M in methanol, 0.52 ml, 0.26 mmol). To this solution was then added 6,8-dibromo-2,3,4,5-tetrahydroquinolin-4-one (0.040 g, 0.13 mmol) in methanol (2 ml). The mixture was then warmed under argon and sodium cyanoborohydride (0.025 g, 0.4 mmol) added. The reaction was then refluxed for 40 h, adding more borohydride after 16 h and 24 h, and evaporated to dryness. The residue was purified on SCX cartridges followed by flash chromatography, eluting with 0-8% “10% ammonia in methanol” in dichloromethane, to give the title compound as an off-white gum (0.009 g, 14%); δH (CD₃OD) 1.65-2.0 (4H, m), 2.65-2.8 (2H, m), 3.2-3.4 (4H, m), 3.65-3.75 (1H, m), 5.55 (1H, s), 7.1-7.55 (5H, m), and 7.97 (1H, d); MS (ES+) 505, 507, 509 (15, 30, 15%, MH+) and 218 (100); MS (ES−) 503, 505, 507 (50, 100, 45%, [M−H]⁻).

### EXAMPLE 4

### N-(6,8-DIBROMO-1,2,3,4-TETRAHYDROQUINOLIN-4-YL)-N′-(1H-IMIDAZO [4,5-B]PYRIDINE-2-YL)-PROPANE-1,3-DIAMINE DIHYDROCHLORIDE.

To N-(1H-imidazo[4,5-b]pyridin-2-yl)propane-1,3-diamine (described in Example 8, step c)) (0.055 g, 0.29 mmol) and 6,8-dibromo-2,3,4,5-tetrahydroquinolin-4-one (0.088 g, 0.29 mmol) in methanol (2 ml) and acetic acid (0.06 g) was added sodium cyanoborohydride (0.019 g, 0.3 mmol). The reaction was then refluxed for 20 h. The reaction mixture was applied to a 2 g SCX cartridge which was flushed with MeOH (15 ml). The cartridge was then eluted with 15 ml 0.2 M NH₃ in MeOH, and this eluate evaporated to dryness. Further purification on silica gel eluting with 0-10% (9:1 methanol/0.880 aq. ammonia) in dichloromethane gave the title compound, compound 2, which was converted to its dihydrochloride by dissolution in 1.0 M HCl in methanol (0.4 ml) and the solution evaporated to dryness to give a white solid (0.060 g, 37%); δ_{H} (CD₃OD) 8.0 (1H, dd, J=6.3, 1.2 Hz), 7.9 (1H, dd, J=6.5, 1.2 Hz), 7.55 (1H, d, J=2.2 Hz), 7.4 (1H, d, J=2.2 Hz), 7.25 (1H, dd, J=6.5, 6.3 Hz), 4.5 (1H, bs), 3.7 (2H, t, J=6.6 Hz), 3.65-3.1 (4H, m), 2.4 (1H, m), 2.2-1.95 (3H, m); m/z (ES+) 479 (6%, MH⁺), 192 (100%).

### EXAMPLE 5

### 2-{[(1R, 2S)-2-(3,4-DICHLOROBENZYLAMINO)CYCLOPENTYLMETHYL]AMINO}-1H-QUINOLIN-4-ONE.

MRSi compound 3 was prepared as described in Example 71 of U.S. Pat. No. 6,320,051 which is incorporated by reference herein.

### EXAMPLE 6

### N-(4,5-DIBROMO-3-METHYLTHIOPHEN-2-YLMETHYL)-N′-(1H-QUINOLIN-4-ONE)PROPANE-1,3-DIAMINE MUPIROCINATE.

Using the general method for reductive amination a mixture of 2-(3-aminoprop-1-ylamino)-1H-quinolin-4-one diamine (J. Med. Chem. 2002, 45, 1959) and 4,5-dibromo-3-methylthiophene-2-carbaldehyde gave N-(4,5-Dibromo-3-methylthiophen-2-ylmethyl)-N′-(1H-quinolin-4-one)propane-1,3-diamine 4 as a white solid (0.034 g, 49%). m/z (ES+) 484 (100% M+).

Using the general method for Mupirocinate formation (Example 1) a mixture of N-(4,5-Dibromo-3-methylthiophen-2-ylmethyl)-N′-(1H-quinolin-4-one)propane-1,3-diamine 4 and Psuedomonic acid A gave the title compound as an off-white solid (0.008 g). mp. 60-70 C.; −δ_{H} (CD₃OD) 0.95 (3H, d, J=6.8 Hz, CH₃), 1.20 (3H, d, J=6.4 Hz, CH₃), 1.31-1.44 (9H, m), 1.58-1.75 (6H, m), 1.94 (2H, quin., J=6.8 Hz, CH₂), 1.96 (1H, m), 2.18 (3H, s, CH₃), 2.23 (3H, s, CH3), 2.24 (1H, m), 2.26 (2H, t, J=7.2 Hz, CH₂), 2.64 (1H, bd, J=13.6 Hz), 2.71 (1H, dd, J=2.2, 7.4 Hz, CH), 2.81 (1H, m, CH), 2.91 (2H, t, J=6.8 Hz, CH₂), 3.36 (1H, dd, J=3.2, 8.8 Hz, CH), 3.40 (2H, t, J=6.6 Hz, CH₂), 3.56 (1H, bd, J=11.6 Hz, CH), 3.72-3.87 (4H, m), 4.07 (2H, t, J=6.8 Hz, CH₂), 4.09 (2H, s, CH₂), 5.66 (1H, s, ArH), 5.74 (1H, bs, CH), 7.24 (1H, td, J=0.8, 7.6 Hz, ArH), 7.38 (1H, d, J=8.1 Hz, ArH), 7.52 (1H, td, J=1.6, 8.1 Hz, ArH) 8.07 (1H, d, J=7.6 Hz, ArH)

### EXAMPLE 7

### N-(4-BROMO-5-(1-FLUOROVINYL)-3-METHYLTHIOPHEN-2-YLMETHYL)-N′-(1H-QUINOLIN-4-ONE)PROPANE-1,3-DIAMINE MUPIROCINATE.

a) Diphenyl(1-fluorovinyl)methylsilane. In a flame-dried 1 L 3-neck round bottom under an anhydrous atmosphere, 65 mL (309 mmol) of diphenylmethylchlorosilane was added to 4.3 g (618 mmol) of lithium wire in 650 mL of anhydrous THF. The mixture was stirred at ambient temperature for 20 hours. The mixture was then cooled to −78° C. and the atmosphere replaced with 1,1-difluoroethylene (excess) such that the temperature of the reaction mixture remained below −55° C. Difluoroethylene addition was stopped when the reaction temperature remained at or below −70° C. The reaction was stirred at <−70° C. until it turned a clear light yellow (˜2 hr.) and was then allowed to warm to ambient temperature. The remaining lithium wire was removed and the mixture treated with portions of Na₂SO₄-10H₂O until no gas evolved upon addition. The mixture was then dried over Na₂SO₄, filtered through a silica pad and the pad rinsed with ether. The combined filtrates were dried under vacuum, the resulting residue suspended/dissolved in hexanes and filtered through another silica pad. The pad was rinsed with hexanes, the filtrates combined and the solvent removed under reduced pressure to give a light yellowish liquid with some white crystalline material present. The product was purified by vacuum distillation (113-117° C. at ˜2 Torr) to give 44 g (59%) of the title compound as a clear colorless liquid. δ_{H} (CDCl₃): 0.72 (3H, s, CH₃), 4.85 (1H, dd, J=2.6, 61.2 Hz, CH₂), 5.48 (1H, dd, J=2.6, 33.3, CH₂), 7.39 (6H, m, ArH), 7.59 (4H, d, J=6.8 Hz, ArH); δ_{F} (CDCl₃): -103.16 (q, dd, J=33.3, 61.2 Hz).

b) 4-Bromo-5-(1-fluorovinyl)-3-methylthiophene-2-carbaldehyde. Under an inert atmosphere in a 25 mL round bottom flask were combined 166 mg of diphenyl(1-fluorovinyl)methylsilane from a) (0.685 mmol), 130 mg of 4,5-dibromo-3-methylthiophene-2-carbaldehyde (0.459 mmol), 209 mg of CsF (1.38 mmol), 88 mg of CuI (0.459 mmol), 10.5 mg Pd₂(dba)₃ (0.0115 mmol) and 14.1 mg AsPh₃ (0.0459 mmol). The flask containing the solids was cooled to 0° C. with an ice bath and 2 mL of degassed, anhydrous dimethylformamide (DMF) were added. The reaction mixture was stirred at ˜0 to 5° C. for 2 hr and then 2 mL of water was added. The mixture was then diluted with 5 mL 1N NaOH and extracted with 25% diethyl ether/hexanes (4×20 mL). The combined extracts were washed with brine (1×5 mL), dried over Na₂SO₄, and the solvent removed under vacuum. The remaining residue was purified by flash silica gel chromatography (CH₂Cl₂/hexanes) to give a 50% yield of the title compound as a white solid. δ_{H} (CDCl₃) 2.57 (3H, s, CH₃), 5.24 (1H, dd, J=4.0, 18.5 Hz, CH₂), 5.70 (1H, dd, J=4.0, 49.6 Hz, CH₂), 10.06 (1H, s, CHO); δ_{F} (CDCl₃): −92.20 (q, dd, J=18.4, 50.4 Hz); m/z (ESI⁺) (MH⁺, 249).

c) N-(4-bromo-S-(1-fluorovinyl)-3-methylthiophen-2-ylmethyl)-N′(1H-quinolin-4-one)propane-1,3-diamine. Under a dry atmosphere and at ambient temperature, 1.00 g (3.45 mmol) of 2-(3-aminoprop-1-ylamino)-1H-quinolin-4-one dihydrochloride and 0.770 g (9.39 mmol) of NaOAc were dissolved in 40 mL of anhydrous MeOH and stirred for 10 min. at ambient temperature. 0.780 g of 4-bromo-5-(1-fluorovinyl)-3-methylthiophene-2-carbaldehyde from b) (3.13 mmol) was then added followed by 8 mL of trimethylorthoformate and an additional 10 mL of anhydrous MeOH. The mixture was stirred at ambient temperature for 2 hr. The solvent was then removed under reduced pressure and the remaining residue was dissolved in 50 mL anhydrous MeOH and 0.474 g (12.5 mmol) of NaBH₄ was added at ambient temperature with stirring. After stirring for 30 min. at ambient temperature, the solvent was removed under reduced pressure and the resulting gummy solid was triturated with 0.1N NaOH (1×, stirring overnight required for product to solidify), deionized water (2×) and 1:1 Et₂O/Hexanes (2×). The remaining solid was dried under vacuum and the product purified by flash silica gel chromatography (NH₃ saturated MeOH/CH₂Cl₂) to give 900 mg (64%) of the desired product, compound 5, as a white foam. δ_{H} (CD₃OD/CDCl₃) 1.82 (2H, quin., J=6.4 Hz, CH₂), 2.15 (3H, s, CH₃), 2.74 (2H, t, J=6.4 Hz, CH₂), 3.33 (2H, t, J=6.8 Hz, CH₂), 3.92 (2H, s, CH₂), 4.94 (1H, dd, J=3.8, 18.6 Hz, CH₂), 5.33 (1H, dd, J=3.8, 50.6 Hz, CH₂), 5.58 (1H, s, CH), 7.18 (1H, d, J=8.0 Hz, ArH), 7.20 (1H, ddd, J=1.2, 7.1, 8.0, ArH), 7.45 (1H, ddd, J=1.4, 7.1, 8.3 Hz, ArH) 8.07 (1H, dd, J=1.2, 8.3 Hz, ArH); m/z (ESI⁺) (MH⁺, 450).

d) N-(4-bromo-5-(1-fluorovinyl)-3-methylthiophen-2-ylmethyl)-N′(1H-quinolin-4-one)propane-1,3-diamine Mupirocinate. Using the general method for Mupirocinate formation (Example 1) a mixture of N-(4-bromo-5-(1-fluorovinyl)-3-methylthiophen-2-ylmethyl)-N′-(1H-quinolin-4-one)propane-1,3-diamine 5 from c) and Psuedomonic acid A gave the title compound as an off-white solid (2.1 g). mp. 65-200 C. (decomposition greater than 200 C.) δ_{H} (CD₃OD/d₆-DMSO) 0.90 (3H, d, J=7.2 Hz, CH₃), 1.15 (3H, d, J=6.4 Hz, CH₃), 1.31-1.40 (9H, m), 1.54-1.70 (6H, m), 1.84 (2H, quin., J=7.0 Hz, CH₂), 1.90 (1H, m), 2.16 (3H, s, CH₃), 2.18 (3H, s, CH₃), 2.22 (1H, m), 2.23 (2H, t, J=7.2 Hz, CH₂), 2.60 (1H, m), 2.67 (1H, dd, J=2.0, 7.6 Hz, CH), 2.77 (2H, t, J=6.8 Hz, CH₂), 2.78 (1H, m, CH), 3.29 (1H, dd, J=2.8, 9.2 Hz, CH), 3.36 (2H, t, J=6.8 Hz, CH₂), 3.50 (1H, bd, J=11.6 Hz, CH), 3.65-3.82 (4H, m), 3.98 (2H, s, CH₂), 4.04 (2H, t, J=6.8 Hz, CH₂), 5.03 (1H, dd, J=4.0, 18.8 Hz, CH₂), 5.35 (1H, dd, J=3.8, 50.4 Hz, CH₂), 5.56 (1H, s, ArH), 5.71 (1H, bs, CH), 7.22 (1H, ddd, J=1.0, 7.5, 8.2 Hz, ArH), 7.38 (1H, d, J=7.3 Hz, ArH), 7.52 (1H, ddd, J=1.2, 7.3, 8.2 Hz, ArH) 8.04 (1H, dd, J=1.2, 7.5 Hz, ArH); δ_{F} (CD₃OD/d₆-DMSO): −92.60 (uncalibrated) (dd, J=18.8, 50.4 Hz); m/z (ESI⁺) (MH⁺, 450).

### EXAMPLE 8

### N-(4-BROMO-5-(1-FLUOROVINYL)-3-METHYLTHIOPHEN-2-YLMETHYL)-N′-(1H-IMIDAZO [4,5-B]PYRIDIN-2-YL)-PROPANE-1,3-DIAMINE.

a) 1,3-Dihydroimidazo[4,5-b]pyridine-2-thione To 2,3-diaminopyridine (4.36 g, 40 mmol) in pyridine (40 ml) was added carbon disulfide (3.6 ml, 60 mmol). The mixture was heated to 50° C. for 6 h then concentrated to low volume by evaporation under reduced pressure and the residue triturated with tetrahydrofuran. The pale brown solid was collected by filtration and dried to give a first crop of 3.6 g. A second crop (2.44 g) was obtained from the filtrate by re-evaporation and trituration with tetrahydrofuran. m/z (ESI+) 152 (MH⁺, 100%).

b) 2-Methanesulfanyl-1H-imidazo[4,5-b]pyridine To the compound from step a) (5.55 g, 36.75 mmol) in dry tetrahydrofuran (100 ml) under argon was added triethylamine (5.66 ml, 40 mmol) and iodomethane (2.5 ml, 40 mmol). After stirring for 20 h at 20° C. the solid was removed by filtration and washed with THF. The combined filtrates were evaporated to dryness and triturated with dichloromethane. The solid was collected by filtration, (4.55 g, 75%). m/z (ESI+) 166 (MH⁺, 100%).

c) N-(1H-imidazo[4,5-b]pyridin-2-yl)propane-1,3-diamine. The product from step b) (4.55 g) was treated with 1,3-diaminopropane (40 ml) at reflux under argon for 50 h. The solvent was removed by evaporation under reduced pressure and the residue triturated with diethyl ether to give a brown solid. This was purified by chromatography on silica gel eluting with 5-25% (9:1 methanol/0.880 aq. ammonia) in dichloromethane to give the required product, (2.6 g, 50%)

d) General method for reductive amination. To a suspension of the amine (0.2 mmol) (containing 0.5 mmol sodium acetate if the amine was present as the dihydrochloride) in methanol (2 ml) was added the aldehyde (0.2 mmol) in methanol (2 ml) and acetic acid (0.033 ml). After stirring under argon for 10 min, NaCNBH₃ (24 mg, 0.4 mmol) in MeOH (1 ml) was added and the reaction stirred for 16 h. The reaction mixture was applied to a 2 g Varian Bond Elute SCX cartridge which was flushed with MeOH (8 ml). The cartridge was then eluted with 8 ml 0.2 M NH₃ in MeOH, and this eluate evaporated to dryness. The residue was purified by chromatography on silica gel eluting with 2-10% (9:1 MeOH/20 M NH₃) in CH₂Cl₂. Product-containing fractions were combined and evaporated under reduced pressure to give the product as a white solid. To convert this into the corresponding dihydrochloride, the solid was dissolved in 1.0 M HCl in methanol (0.4 ml) and the solution evaporated to dryness.

e) N-(4-bromo-5-(1-fluorovinyl)-3-methylthiophen-2-ylmethyl)-N′-(1H-imidazo[4,5-b]pyridin-2-yl)-propane-1,3-diamine. Using the general method for reductive amination a mixture of N-(1H-imidazo[4,5-b]pyridin-2-yl)propane-1,3-diamine from step c) and 4-Bromo-5-(1-fluorovinyl)-3-methylthiophene-2-carbaldehyde as prepared in Example 7b) gave the title compound as a white solid. m/z (ES+) 424 (100% M+).

### EXAMPLE 9

### N-(3-CHLORO-5-METHOXY-1H—INDOL-2-YLMETHYL)-N′-(1H-IMIDAZO[4,5-B]PYRIDIN-2-YL)-PROPANE-1,3-DIAMINE MUPIROCINATE.

a) 5-Methoxyindoline-7-carbaldehyde. 1-(tert-Butoxycarbonyl)-5-methoxyindoline (Heterocycles, 1992, 34, 1031; 1.75 g 7.0 mmol) was dissolved in dry THF, treated with TMEDA (1.4 ml) and cooled to −78° C. under an argon atmosphere. A solution of s-butyl lithium (1.3 M in cyclohexane, 5.18 ml) was added dropwise. After stirring at −78° C. for 1 h, the solution was treated with dry DMF (1.08 ml, 14 mmol) and stirred for a further 0.5 h. The cooling bath was then removed and the solution allowed to reach room temperature over 1 h. The reaction mixture was quenched with 10% aqueous NH₄Cl and the product extracted into ethyl acetate. The extracts were combined, washed with water and brine, dried (MgSO₄) and evaporated. The residue was chromatographed on Kieselgel 60 eluting with 0-20% ethyl acetate in hexane. Product-containing fractions were combined and evaporated to afford the title compound (510 mg); contaminated with 35% (by weight) of the corresponding N-Boc analogue; δ_{H} (CDCl₃, inter alia) 3.03 (2H, t, J=8.0 Hz, CH₂), 3.76 (2H, t, J=8.1 Hz, CH₂NH), 3.77 (3H, s, OMe), 6.42 (1H, br.s, NH), 6.73 (1H, d, J=0.8 Hz Ar—H), 6.90-6.92 (1H, m, Ar—H), 9.79 (1H, s, CHO).

b) 5-Methoxyindole-7-carbaldehyde. The product from 9a (80 mg; containing 0.3 mmol 5-methoxyindoline-7-carbaldehyde) was dissolved in dichloromethane (10 ml) and treated with MnO₂ (344 mg, 4.0 mmol). The reaction mixture was stirred at room temperature for 16 h, filtered through Celite and the solvent removed in vacuo. The residue was chromatographed on Kieselgel 60 eluting with 0-20% ethyl acetate in hexane to afford the title compound as a pale yellow solid (23 mg, 44%), δ_{H} (CDCl₃) 3.91(3H, s, OMe), 6.56 (1H, dd, J=2.2, 3.2 Hz, 3-H), 7.28 (1H, d, J=2.3 Hz, Ar—H), 7.33(1H, t, J=2.6 Hz, 2-H), 7.46(1H, m, Ar—H), 9.93(1H, br.s., NH), 10.07 (1H, s, CHO).

c) 3-Chloro-5-methoxy-1H-indol-7-carbaldehyde. 5-Methoxyindole-7-carbaldehyde from b) (40 mg, 0.22 mmol) was dissolved in dichloromethane (5 ml), treated with N-chlorosuccinimide (40 mg), and the mixture stirred at room temperature for 16 h. The solution was then diluted with dichloromethane, washed with water and brine, dried (MgSO₄) and evaporated to a pale brown solid.

d) N-(3-Chloro-5-methoxy-1H-indol-7-ylmethyl)-N′(1H-imidazo[4,5-b]pyridin-2-yl)-propane-1,3-diamine. The product from c) was coupled to the N-(1H-imidazo[4,5-b]pyridin-2-yl)propane-1,3-diamine from Example 8, step c) on a 0.2 mmol scale using the general method for reductive amination (Example 8, step d) to give the title compound, compound 7, as a white solid (7 mg, 9%); m/z (CI⁺) 386 (MH⁺, 70%).

e) N-(3-Chloro-5-methoxy-1H-indol-2-ylmethyl)-N′-(1H-imidazo[4,5-b]pyridin-2-yl)-propane-1,3-diamine Mupirocinate. Using the general method for Mupirocinate formation (Example 1) a mixture of N-(3-Chloro-5-methoxy-1H-indol-7-ylmethyl)-N′-(1H-imidazo[4,5-b]pyridin-2-yl)-propane-1,3-diamine, compound 7, from d) and Psuedomonic acid A gave the title compound as an off-white solid (0.010 g). mp. 86-88 C.

### EXAMPLE 10

### N-(1H-IMIDAZO [4,5-B]PYRIDIN-2-YL)-N′-(3,4,6-TRICHLORO-1H—INDOL-2-YLMETHYL)-PROPANE-1,3-DIAMINE MUPIROCINATE.

a) N-(3,4,6-Trichloro-1H-indol-2-ylmethyl)-N′-(1H-imidazo[4,5-b]pyridin-2-yl)-propane-1,3-diamine. 3,4,6-Trichloroindole-2-carboxaldehyde was coupled to the compound from Example 8, step c) on a 0.1 mmol scale using the general method for reductive amination to give the title compound as a white solid (15 mg, 35%); ¹H NMR δ_{H} (CD₃OD/CDCl₃) 1.85 (2H, quin., J=6.8 Hz, CH₂), 2.71 (2H, t, J=6.8 Hz, CH₂), 3.46 (2H, t, J=6.8 Hz, CH₂), 3.92 (2H, s, CH₂), 6.94 (1H, dd, J=5.2, 7.6 Hz, ArH), 6.99 (1H, d, J=1.8 Hz, ArH), 7.21 (1H, d, J=1.8, ArH), 7.42 (1H, d, J=7.6 Hz, ArH) 7.92 (1H, d, J=5.2 Hz, ArH); m/z (ESI⁺) 422 (MH⁺).

b) Using the general method for Mupirocinate formation (Example 1) a mixture of N-(1H-imidazo[4,5-b]pyridin-2-yl)-N′-(3,4,6-trichloro-1H-indol-2-ylmethyl)—propane-1,3-diamine 8 (from Example 10a) and Psuedomonic acid A gave the title compound as an off-white solid (0.011 g). mp. 96-98 C.

### EXAMPLE 11

### N-(1H-IMIDAZO[4,5-B] PYRIDIN-2-YL)-N′-(3,4,6-TRICHLORO-1H-INDOL-2-YLMETHYL)-PROPANE-1,3-DIAMINE FUSIDATE.

Using the general method for Fusidate formation (Example 2) a mixture of N-(1H-imidazo[4,5-b]pyridin-2-yl)-N′-(3,4,6-trichloro-1H-indol-2-ylmethyl)—propane-1,3-diamine 8 (from Example 10a) and Fusidic acid gave the title compound as an off-white solid (0.01 g). mp. 153-158 C. ¹H NMR δ_{H} (CD₃OD) 0.88 (3H, d, J=6.8 Hz, CH₃), 0.93 (3H, s, CH₃), 0.98 (3H, s, CH₃), 1.12 (2H, m), 1.2 (1H, d, J=14.0 Hz, CH), 1.37 (3H, s, CH₃), 1.44-2.38 (16H, m), 1.59 (3H, s, CH₃), 1.65 (3H, s 3H), 1.94 (3H, s, CH₃), 1.98 (2H, quin., J=6.4 Hz, CH₂), 2.53 (1H, m), 3.03 (2H, t, J=6.4 Hz, CH₂), 3.53 (2H, t, J=6.0 Hz, CH₂), 3.64 (1H, bd, J=2.4 Hz, CH), 4.21 (2H, s, CH₂), 4.29 (1H, s, CH), 5.13 (1H, t, J=7.0, CH), 5.80 (1H, d, J=8.4 Hz, CH), 7.03 (1H, dd, J=5.1, 7.8 Hz, ArH), 7.10 (1H, d, J=1.8 Hz, ArH), 7.42 (1H, d, J=1.8, ArH), 7.51 (1H, dd, J=1.1, 7.8 Hz, ArH) 8.08 (1H, dd, J=1.1, 5.1 Hz, ArH).

### EXAMPLE 12

### MRS Inhibitors Alone and in Combination with Mupirocin are Active Against Multiresistant S. Aureus.

The MRS inhibitors,

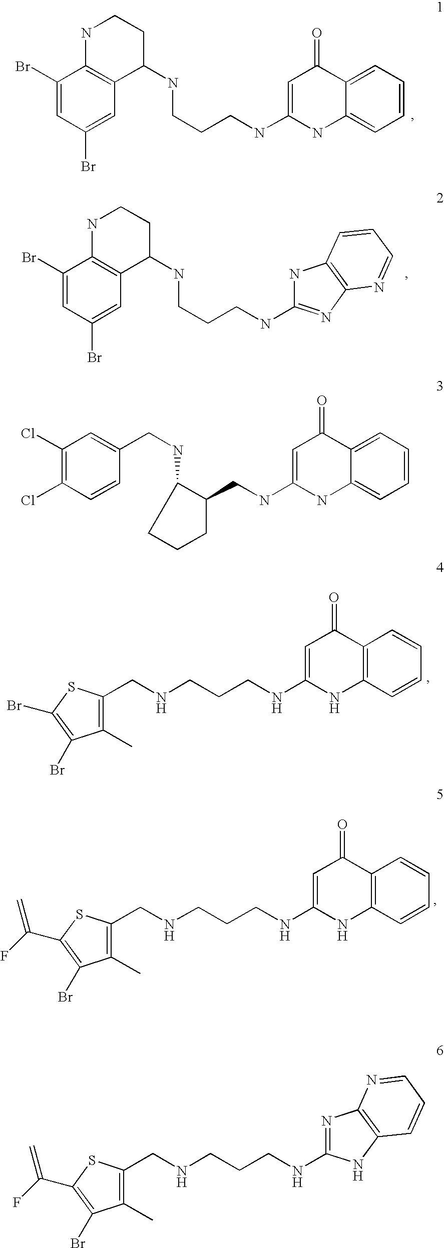

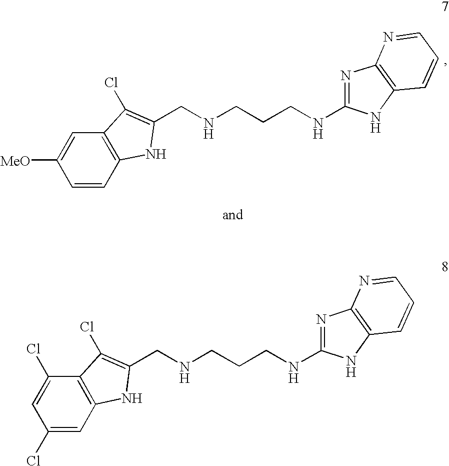

were tested against a collection of clinical isolates of S. aureus including strains that were multi-resistant to mupirocin and to other agents such as gentamicin and oxacillin. The results in Table 1 show that the antibacterial activities of all three MRS inhibitors were not affected by cross-resistance to other drug classes. For example, MRSi compound 2 demonstrated equivalent activity (MICs ranging from 0.06-0.25 μg/mL) against all strains tested including those with low and high-level resistance to mupirocin. These data indicate that compounds that inhibit bacterial methionyl tRNA synthetases may have potential for the therapy of infections caused by mupirocin-resistant staphylococci.

TABLE 1
Activity of MRS Inhibitors vs. Mupirocin-Susceptible and -Resistant S. aureus

| | MIC (μg/mL) | | | | | |
|---|---|---|---|---|---|---|
| | MRSi cmpd 1 | MRSi cmpd 2 | MRSi cmpd 3 | Mupirocin | Gentamicin | Oxacillin |
| S. aureus Oxford | 0.12 | 0.12 | 2 | 0.12 | 0.5 | 0.015 |
| S. aureus ATCC | 0.25 | 0.25 | 4 | 0.12 | 4 | 4 |
| 43300 (ORSA) | | | | | | |
| S. aureus NRS1 | 0.12 | 0.25 | 4 | 0.12 | >64 | >64 |
| (VISA) | | | | | | |
| S. aureus NRS107 | 0.03 | 0.06 | 1 | >64 | 0.25 | 0.12 |
| (HL Mup rest.) | | | | | | |
| S. aureus LZ1 (HL | 0.06 | 0.12 | 2 | >64 | 0.5 | >64 |
| Mup rest.) | | | | | | |
| S. aureus LZ6 (HL | 0.12 | 0.25 | 2 | >64 | 0.25 | >64 |
| Mup rest.) | | | | | | |
| S. aureus LZ8 (LL | 0.06 | 0.25 | 4 | 32 | 64 | >64 |
| Mup. Rest.) | | | | | | |
| S. aureus LZ9 | 0.06 | 0.25 | 2 | 0.25 | 64 | >64 |
| S. aureus LZ10 | 0.12 | 0.25 | 2 | 0.25 | 64 | >64 |
| S. aureus 101-100 | 0.06 | 0.12 | 2 | <0.06 | 0.12 | 16 |
| (Mup. Susc.) | | | | | | |
| S. aureus 10-420 | 0.12 | 0.25 | 2 | >64 | 64 | 16 |
| (LL Mup. Rest.) | | | | | | |
| S. aureus 14-354 | 0.25 | 0.25 | 4 | 64 | >64 | >64 |
| (LL Mup. Rest.) | | | | | | |
| S. aureus 25-670 | 0.12 | 0.25 | 4 | >64 | >64 | >64 |
| (HL Mup. Rest.) | | | | | | |
| S. aureus 31-1334 | 0.12 | 0.25 | 2 | 32 | 64 | >64 |
| (LL Mup Rest.) | | | | | | |
| S. aureus 36-1298 | 0.015 | 0.06 | 1 | 32 | 0.5 | 0.25 |
| (LL Mup. Rest.) | | | | | | |
| S. aureus 87-2797 | 0.12 | 0.25 | 2 | 0.25 | 0.25 | >64 |
| (Mup. Susc.) | | | | | | |
| S. aureus 87-2797 | 0.12 | 0.25 | 4 | >64 | 0.25 | >64 |
| (HL Mup. Rest.) | | | | | | |
| S. aureus Miles | 0.12 | 0.12 | 2 | 64 | 0.5 | 0.25 |
| Hall | | | | | | |
| MIC Range | 0.015-0.12 | 0.06-0.25 | 1-4 | <0.06->64 | 0.12->64 | 0.015->64 |
| MIC 90 | 0.25 | 0.25 | 4 | >64 | >64 | >64 |

The MRS inhibitor 4 alone and in combination with mupirocin (mupirocin salt and 1:1 combination) were tested against a collection of Gram-positive pathogens. The results in Table 2 show that both the mupirocin salt of MRSi compound 4 and MRSi compound 4/mupirocin 1:1 combination demonstrated equivalent activity against all the organisms tested. The combination products demonstrated potent activity against mupirocin-resistant S. aureus and S. epidermidis.

TABLE 2
Antibacterial activity MRS inhibitor 4 alone and in combination with
mupirocin

| | MIC (μg/mL) | | | |
|---|---|---|---|---|
| | MRSI Cmpd | | MRSi Cmpd | |
| | 4 | MRSi Cmpd 4 | 4/mupirocin | |
| | acetate | mupirocinate | 1:1 combination | Mupirocin |
| Structure | 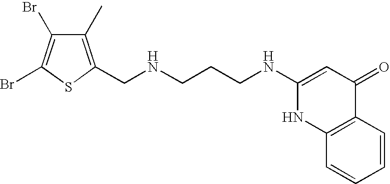 | | | 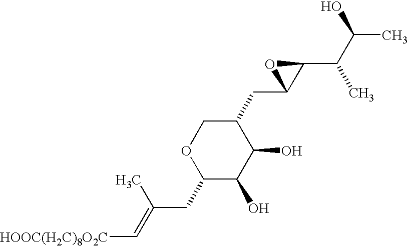 |
| S. aueus | 0.25 | 0.5 | 0.12/0.12 | 0.12 |
| ATCC | | | | |
| 29213 | | | | |
| S. aureus | 0.03 | 0.25 | 0.06/0.06 | 0.06 |
| Oxford | | | | |
| S. aureus | 0.03 | 0.25 | 0.25/0.25 | >8 |
| NRS107 | | | | |
| (mupA) | | | | |
| S. aureus | 0.25 | 0.5 | 0.12/0.12 | 0.25 |
| ATCC | | | | |
| 43300 (ORSA) | | | | |
| E. faecalis 1 | 0.015 | 0.12 | 0.03/0.03 | 8 |
| E. faecalis 7 | ≦0.008 | 0.12 | 0.06/0.06 | 8 |
| E. faecalis | ≦0.008 | 0.06 | 0.06/0.06 | 8 |
| ATCC | | | | |
| 29212 | | | | |
| S. pyogenes | 0.25 | 0.25 | 0.25/0.25 | 0.12 |
| ATCC | | | | |
| 19615 | | | | |
| S. pyogenes | 0.12 | 0.25 | 0.12/0.12 | 0.12 |
| MB143 | | | | |
| (macrolide | | | | |
| rest.) | | | | |
| S. epidermidis | 0.5 | 1 | 1/1 | >8 |
| NRS6 | | | | |
| S. epidermidis | 0.03 | 0.25 | 0.12/0.12 | 8 |
| NRS7 | | | | |
| S. epidermidis | 0.25 | 1 | 0.25/0.25 | >8 |
| NRS8 | | | | |
| S. hemolyticus | 0.25 | 0.5 | 0.12/0.12 | 0.12 |
| NRS50 | | | | |
| S. hemolyticus | 0.5 | 0.5 | 0.25/0.25 | 0.25 |
| NRS116 | | | | |

The MRS inhibitor 8 was also prepared as a fusidate and mupirocinate salt and tested for antibacterial activity against Gram-positive bacteria (Table 3). The mupirocinate and fusidate salts demonstrated equivalent antibacterial activities against all the organisms tested. MRSi compound 8 alone and the fusidate and mupirocin salts were active against mupirocin-resistant S. aureus and S. epidermidis and organisms such as E. faecalis that are not susceptible to mupirocin.

TABLE 3
Activity of the acetate, fusidate and mupirocinate salts of the MRS inhibitor 8
against Gram-positive bacteria

| | MIC (μg/mL) | | | |
|---|---|---|---|---|
| | | MRSi | MRSi Cmpd | MRSi Cmpd |
| | | Cmpd 8 | 8 | 8 |
| | Mupirocin | acetate | fusidate | mupirocinate |
| Structure | 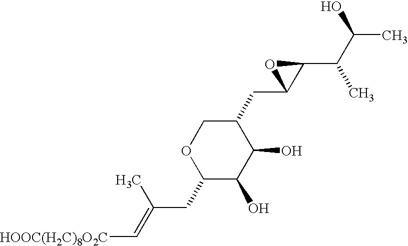 | 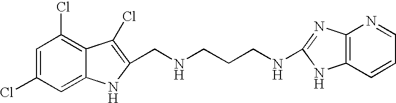 | | |
| S. aueus | 0.12 | 0.06 | 0.12 | 0.12 |
| ATCC | | | | |
| 29213 | | | | |
| S. aureus | 0.06 | 0.03 | 0.06 | 0.12 |
| Oxford | | | | |
| S. aureus | >8 | 0.03 | 0.06 | 0.12 |
| NRS107 | | | | |
| (mupA) | | | | |
| S. aureus | 0.25 | 0.03 | 0.06 | 0.12 |
| ATCC | | | | |
| 43300 | | | | |
| (ORSA) | | | | |
| E. faecalis 1 | 8 | 0.03 | 0.06 | 0.06 |
| E. faecalis 7 | 8 | 0.03 | 0.12 | 0.12 |
| E. faecalis | 8 | 0.03 | 0.12 | 0.12 |
| ATCC | | | | |
| 29212 | | | | |
| S. pyogenes | 0.12 | 0.12 | 0.5 | 0.25 |
| ATCC | | | | |
| 19615 | | | | |
| S. pyogenes | 0.12 | 0.12 | 0.5 | 0.12 |
| MB143 | | | | |
| (macrolide | | | | |
| rest.) | | | | |
| S. epi- | >8 | 0.12 | 0.25 | 0.5 |
| dermidis | | | | |
| NRS6 | | | | |
| S. epi- | 8 | 0.06 | 0.12 | 0.12 |
| dermidis | | | | |
| NRS7 | | | | |
| S. epi- | >8 | 0.12 | 0.25 | 0.25 |
| dermidis | | | | |
| NRS8 | | | | |
| S. hemo- | 0.12 | 0.06 | 0.12 | 0.12 |
| lyticus | | | | |
| NRS50 | | | | |
| S. hemo- | 0.25 | 0.12 | 0.25 | 0.25 |
| lyticus | | | | |
| NRS116 | | | | |

The MRS inhibitor 5 acetate and mupirocin salts were tested against a collection of Gram-positive bacteria. In common with the other MRS inhibitors, MRSi compound 5 alone and in combination with mupirocin demonstrated potent activity against all pathogens including mupirocin and oxacillin (methicillin) resistant S. aureus as shown in Table 4.

TABLE 4
Activity of the acetate and mupirocin salts of the MRS inhibitor 5 against
Gram-positive bacteria

| | MIC (μg/mL) | | |
|---|---|---|---|
| | MRSi Cmpd | | |
| | 5 | MRSi Cmpd 5 | |
| | acetate | mupirocinate | Mupirocin |
| Structure | 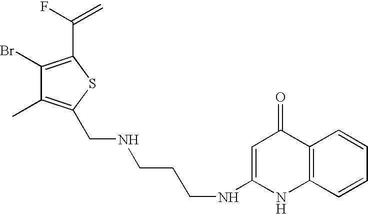 | | 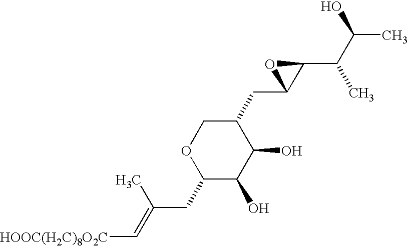 |
| S. aueus ATCC 29213 | 0.06 | 0.06/0.06 | 0.12 |
| S. aureus Oxford | ≦0.008 | 0.015/0.015 | 0.06 |
| S. aureus | ≦0.008 | 0.008/0.008 | >8 |
| NRS107 (mupA) | | | |
| S. aureus | 0.25 | 0.12/0.12 | 0.12 |
| ATCC 43300 | | | |
| (ORSA) | | | |
| E. faecalis 1 | 0.004 | ≦0.004/≦0.004 | >8 |
| E. faecalis 7 | 0.015 | ≦0.004/≦0.004 | >8 |
| E. faecalis | 0.015 | 0.008/0.008 | >8 |
| ATCC 29212 | | | |
| S. pyogenes | 0.12 | 0.12/0.12 | 0.12 |
| ATCC 19615 | | | |
| S. pyogenes | 0.12 | 0.03/0.03 | 0.12 |
| MB000143 | | | |
| S. epidermidis | 0.25 | NT | >8 |
| NRS6 | | | |
| S. epidermidis | 0.06 | 0.015/0.015 | 8 |
| NRS7 | | | |
| S. epidermidis | 0.12 | NT | >8 |
| NRS8 | | | |
| S. hemolyticus | 0.12 | NT | 0.12 |
| NRS50 | | | |
| S. hemolyticus | 0.25 | NT | 0.25 |
| NRS116 | | | |

MRSi compound 5 (acetate and mupirocin salts) were further challenged against 62 recent clinical isolates of S. aureus and Table 5 shows potent activity against both oxacillin-susceptible and resistant organisms.

TABLE 5
Activity of MRSi compound 5 and MRSi compound 5/Mupirocin (1:1
Combination) against oxacillin-susceptible and -resistant S. aureus

| | | MIC (μg/mL) | | | |
|---|---|---|---|---|---|
| Test Substance | Strain Panel | Range | Mode | MIC₅₀ | MIC₉₀ |
| MRSi Cmpd 5 | All (62) | ≦0.008 to 1 | 0.06 | 0.03 | 0.12 |
| (acetate salt) | Oxa-S (20) | ≦0.008 to 0.12 | 0.06 | 0.03 | 0.06 |
| | Oxa-R (42) | ≦0.008 to 1 | 0.03 | 0.03 | 0.5 |
| MRSi | All (62) | ≦0.004/≦0.004 | 0.06/0.06 | 0.06/0.06 | 0.12/0.12 |
| Cmpd 5/Mupirocin | | to 0.5/0.5 | | | |
| | Oxa-S (20) | ≦0.004/<0.004 | 0.06/0.06 | 0.06/0.06 | 0.12/0.12 |
| | | to 0.12/0.12 | | | |
| | Oxa-R (42) | ≦0.004/<0.004 | 0.06/0.06 | 0.06/0.06 | 0.12/0.12 |
| | | to 0.5/0.5 | | | |
| Mupirocin | All (62) | 0.08 to >8 | 0.12 | 0.12 | >8 |
| | Oxa-S (20) | 0.06 to >8 | 0.12 | 0.12 | >8 |
| | Oxa-R (42) | 0.03 to >8 | 0.12 | 0.12 | >8 |

### EXAMPLE 13

### Activity of MRSI Compound 5 and MRSI Compound 5/Mupirocin Against Mupirocin-Resistant S. aureus

The acetate and mupirocin salts of the MRS inhibitor compound 5 were tested against a collection of both low and high-level mupirocin resistant clinical isolates of S. aureus (Table 6). The results show that MRSi compound 5 alone and in combination with mupirocin (mupirocin salt) have potent activity against both low and high-level mupirocin-reistant S. aureus.

TABLE 6
Activity of MRSi compound 5 and MRSI compound 5/mupirocin against mupirocin-resistant S. aureus

| | | MIC (μg/mL) | |
|---|---|---|---|
| Organism/phenotype | | MRSi Cmpd 5 acetate 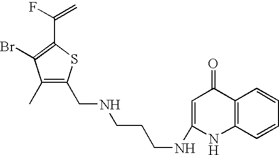 | MRSi Cmpd 5 mipirocinate 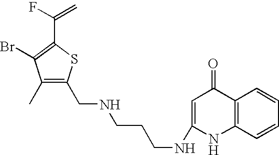 |
| S. aureus (mupirocin- | LZ9 | 0.03 | 0.06/0.06 |
| susceptible) | LZ10 | 0.03 | 0.03/0.03 |
| | 010-100 | 0.03 | 0.03/0.03 |
| | 087-2789 | 0.06 | 0.06/0.06 |
| Low-level mupirocin- | LZ8 | 0.03 | 0.06/0.06 |
| resistant S. aureus (MICs | Miles | ≦0.008 | 0.06/0.06 |
| 8-256 μg/mL) | Hall | | |
| | 014-354 | 0.015 | 0.06/0.06 |
| | 031-1334 | 0.015 | 0.03/0.03 |
| | 036-1298 | ≦0.008 | 0.015/0.015 |
| | 1081148 | 0.5 | 0.5/0.5 |
| | NRS127 | 0.5 | 0.5/0.5 |
| High-level mupirocin- | NRS107 | ≦0.008 | 0.008/0.008 |
| resistant S. aureus (MICs | LZ1 | 0.015 | 0.03/0.03 |
| ≧512 μg/mL) | LZ6 | 0.06 | 0.03/0.03 |
| | 010-420 | 0.03 | 0.06/0.06 |
| | 87-2797 | 0.03 | 0.06/0.06 |
| | 25-670 | 0.03 | 0.03/0.03 |
| | 1079101 | 0.06 | 0.12/0.12 |
| | NRS54 | 0.06 | 0.06/0.06 |

| | | |
|---|---|---|
| MIC Range | ≦0.008-0.5 | 0.008/0.008-0.5/0.5 |
| MIC₅₀ | 0.03 | 0.12 |
| MIC₉₀ | 0.5 | 1 |

| | | MIC (μg/mL) | |
|---|---|---|---|
| Organism/phenotype | | Mupirocin 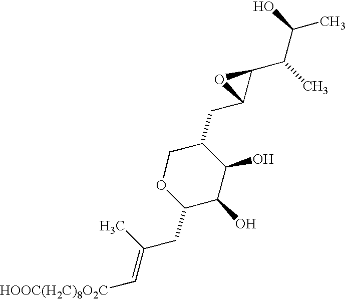 | Oxacillin |
| S. aureus (mupirocin- | LZ9 | 0.12 | >64 |
| susceptible) | LZ10 | 0.12 | >64 |
| | 010-100 | 0.06 | 8 |
| | 087-2789 | 0.12 | >64 |
| Low-level mupirocin- | LZ8 | 16 | >64 |
| resistant S. aureus (MICs | Miles | 32 | 0.12 |
| 8-256 μg/mL) | Hall | | |
| | 014-354 | 16 | >64 |
| | 031-1334 | 16 | 64 |
| | 036-1298 | 16 | 8 |
| | 1081148 | 16 | 8 |
| | NRS127 | 32 | 64 |
| High-level mupirocin- | NRS107 | >256 | 0.12 |
| resistant S. aureus (MICs | LZ1 | >256 | >64 |
| ≧512 μg/mL) | LZ6 | >256 | >64 |
| | 010-420 | >256 | 8 |
| | 87-2797 | >256 | >64 |
| | 25-670 | >256 | >64 |
| | 1079101 | >256 | 0.25 |
| | NRS54 | >256 | >64 |

| | | |
|---|---|---|
| MIC Range | ≦0.06->256 | 0.12->64 |
| MIC₅₀ | 32 | >64 |
| MIC₉₀ | >256 | >64 |

### EXAMPLE 14

### Activity of the MRS Inhibitor 5 Alone MRSi Compound 5/Mupirocin Against Vancomycin-Intermediate S. aureus (VISA)

The acetate and mupirocin salts of MRSi compound 5 were tested against 8 isolates of S. aureus that were vancomycin-intermediate (vancomyin MICs, 8-16 μg/mL). Both MRSi compound 5 alone (acetate) and in combination with mupirocin (mupirocinate) maintained activity against all VISA isolates with MICs ranging from ≦0.008-0.25 μg/mL. In contrast mupirocin demonstrated poor activity against three isolates with MICs that were >8 μg/mL.

TABLE 7
Activity of MRSi compound 5 and MRSi compound 5/mupirocin (1:1
combination) against vancomycin-intermediate S. aureus (VISA)

| | MIC (μg/mL) | | | |
|---|---|---|---|---|
| | MRSi Cmpd 5 | MRSi Cmpd 5 | | |
| Organism | acetate | mupirocate | Mupirocin | Oxacillin |
| S. aureus NRS1 (Mu50) | 0.015 | 0.06/0.06 | 0.06 | >64 |
| S. aureus NRS3 (HIP5827) | 0.008 | 0.03/0.03 | 0.5 | >64 |
| S. aureus NRS49 (Korea) | 0.008 | 0.004/≦0.004 | 0.03 | >64 |
| S. aureus NRS54 (Brazil) | 0.06 | 0.06/0.06 | >8 | >64 |
| S. aureus NRS56 (Brazil) | 0.008 | 0.004/≦0.004 | 0.12 | >64 |
| S. aureus NRS4 (HIP5836) | 0.008 | 0.015/0.015 | 0.12 | 64 |
| S. aureus NRS24 (HIP09143) | 0.06 | 0.12/0.12 | 8 | 32 |
| S. aureus NRS18 (HIP06854) | 0.03 | 0.06/0.06 | 8 | 2 |

### EXAMPLE 15

### Activity of MRS Inhibitors Alone and in Combination with Mupirocin Against Enterococci Including Vancomycin-Resistant Strains

In addition, the MRS inhibitor demonstrated potent activity against the enterococci, including vancomycin-resistant strains (VRE).

MRSi compound 5 alone and in combination with mupirocin was tested recent clinical isolates of E. faecalis (n=28) and E. faecium (n=23). Both the acetate salt and mupirocinate salts of MRSi compound 5 maintained potent activity against vancomycin-susceptible and—resistant enterococci (Tables 8, 9, and 10).

TABLE 8
Activity of MRS Inhibitors against enterococci including
vancomycin resistant strains

| | MIC (μg/mL) | | | | | |
|---|---|---|---|---|---|---|
| | MRSi Cmpd 1 | MRSi Cmpd 2 | MRSi Cmpd 3 | Mupirocin | Ampicillin | Vancomycin |
| E. faecalis 1 | ≦0.004 | 0.015 | 0.12 | 32 | 0.5 | 0.12 |
| E. faecalis 7 | 0.015 | 0.06 | 0.5 | 64 | 0.5 | 0.5 |
| E. faecalis | 0.015 | 0.03 | 0.5 | 32 | 0.5 | 4 |
| ATCC 51299 | | | | | | |
| (VRE) | | | | | | |
| E. faecium | ≦0.004 | ≦0.004 | 0.03 | 1 | 1 | 0.5 |
| ATCC 33667 | | | | | | |

TABLE 9
Activity of MRSi compound 5 and MRSi compound 5/Mupirocin (1:1) against
recent clinical isolates of E. faecalis

| | | Range | Mode | MIC₅₀ | MIC₉₀ |
|---|---|---|---|---|---|
| MRSi Cmpd 5 | | | | | |
| (acetate) | | | | | |
| E faecalis | | | | | |
| | ALL (28) | <0.004-0.015 | <0.004 | <0.004 | 0.015 |
| | Van-S (16) | <0.004-0.015 | ≦0.004 | ≦0.004 | 0.008 |
| | Van-R(11) | <0.004-0.015 | <0.004 | <0.004 | 0.015 |
| MRSi Cmpd 5 | | | | | |
| (mupirocinate) | | | | | |
| | ALL (28) | <0.004/<0.004-0.06/0.06 | 0.03 | 0.008/0.008 | 0.03 |
| | Van-S (16) | ≦0.004/≦0.004-0.06/0.06 | — | 0.008/0.008 | 0.03 |
| | Van-R(11) | <0.004/<0.004-0.06/0.06 | 0.008/0.008-0.0015/0.015 | 0.015/0.015 | 0.03 |
| Mupirocin | | | | | |
| E. faecalis | | | | | |
| | ALL (28) | 2->8 | >8 | >8 | >8 |
| | Van-S (16) | 2->8 | >8 | >8 | >8 |
| | Van-R(11) | 2->8 | >8 | >8 | >8 |

TABLE 10
Activity of MRSi compound 5 and MRSi compound 5/Mupirocin (1:1)
against recent clinical isolates of E. faecium

| | | Range | Mode | MIC₅₀ | MIC₉₀ |
|---|---|---|---|---|---|
| MRSi Cmpd 5 | | | | | |
| (acetate) | | | | | |
| E faecalis | | | | | |
| | ALL (23) | <0.004-0.03 | <0.004 | <0.004 | <0.004 |
| | Van-S (11) | ≦0.004 | ≦0.004 | ≦0.004 | ≦0.004 |
| | Van-R(12) | <0.004-0.03 | <0.004 | <0.004 | <0.004 |
| MRSi Cmpd 5 | | | | | |
| (mupirocinate) | | | | | |
| | ALL (23) | <0.004/<0.004-0.06/0.06 | <0.004/<0.004 | <0.004/<0.004 | 0.008/0.008 |
| | Van-S (11) | ≦0.004/≦0.004-0.008/0.008 | ≦0.004/≦0.004 | ≦0.004/≦0.004 | ≦0.004/≦0.004 |
| | Van-R(12) | <0.004/<0.004-0.06/0.06 | <0.004/<0.004 | <0.004/<0.004 | 0.008/0.008 |
| Mupirocin | | | | | |
| E. faecalis | | | | | |
| | ALL (23) | 0.25->8 | 1 | 1 | >8 |
| | Van-S (11) | 0.25-1 | 1 | 1 | 1 |
| | Van-R(12) | 0.5->8 | 1 | 1 | >8 |

### EXAMPLE 16

### Activity of the MRS Inhibitor MRSi Compound 5 Alone and in Combination with Mupirocin Against Streptococcus pyogenes

S. pyogenes is also a significant skin pathogen and 48 recent clinical isolates were tested for their susceptibility to MRSi compound 5 alone (acetate) and in combination (1:1) with mupirocin (mupirocinate). Both MRSi compound 5 alone and in 1:1 combination showed potent activity against S. pyogenes (Table 11).

TABLE 11
Activity of MRSi compound 5 and MRSi compound 5/Mupirocin
(1:1 Combination) against clinical isolates of S. pyogenes

| | MRSi Cmpd 5 | MRSi Cmpd 5/ | |
|---|---|---|---|
| | (acetate salt) | Mupirocin | Mupirocin |
| MIC Range | 0.03 to 0.25 | 0.03/0.03 to 0.12/0.12 | 0.03-0.5 |
| Mode | 0.06 | 0.06/0.06 | 0.12 |
| MIC₅₀ | 0.06 | 0.06/0.06 | 0.12 |
| MIC₉₀ | 0.12 | 0.12/0.12 | 0.25 |

### EXAMPLE 17

### Synergy Testing: A Methionyl TRNA Synthetase Inhibitor in Combination with Mupirocin

The objective of the study was to determine whether mupirocin (an inhibitor of bacterial isoleucyl tRNA synthetase) demonstrates synergy when combined with MRSi compound 2 (an inhibitor of bacterial methionyl tRNA synthetase) against mupirocin susceptible and resistant strains of Staphylococcus aureus.

Structure of Mupirocin (Pseudomonic Acid)

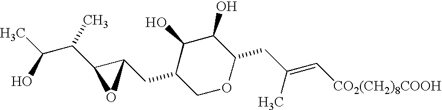

Structure of MRSi Compound 2

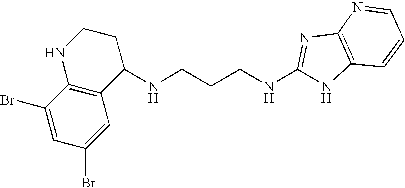

Synergy Testing

The following strains were used in the synergy testing study:

S. aureus ATCC 29213 (mupirocin susceptible)

S. aureus Oxford (mupirocin susceptible)

S. aureus 31-1334 (low level mupirocin resistant clinical isolate)

S. aureus 14-354 (low level mupirocin resistant clinical isolate)

S. aureus 25-670 (high level mupirocin resistant clinical isolate)

The bacterial strains were tested for susceptibility to mupirocin and MRSi compound 2 using the broth microdilution method in accordance with NCCLS guideline to determine their MICs.

The compounds were tested for synergy using the checkerboard method as described by Eliopoulos, G. M., and R. C. Moellering, Jr. 1996. Antimicrobial combinations, p. 330-396. In V. Lorian (ed.), Antibiotics in laboratory medicine. The Williams & Wilkins Co., Baltimore, Md.

Briefly, the MIC and checkerboard titration assays were performed with strains in microtiter trays with cation-supplemented Mueller-Hinton broth (Difco). Inocula were prepared by suspending growth from blood agar plates in sterile saline to a density equivalent to that of a 0.5 McFarland standard and were diluted 1:10 to produce a final inoculum of 5×10⁵ CFU/ml. The trays were incubated aerobically overnight. Standard quality control strains were included with each run. Fractional inhibitory concentrations (FICs) were calculated as the MIC of drug A or B in combination/MIC of drug A or B alone, and the FIC index was obtained by adding the two FICs. FIC indices were interpreted as synergistic if the values were ≦0.5, additive or indifferent if the values were >0.5 to 4, and antagonistic if the values were >4.0. Results are shown in Table 12.

TABLE 12
Activity of mupirocin in combination with MRSi compound 2 (MRS
inhibitor) against mupirocin-susceptible and resistant S. aureus

| | | MRSi cmpd 2 | Mupirocin MIC | | |
|---|---|---|---|---|---|
| Organism | Phenotype | MIC (8 g/mL) | (8 g/mL) | ^{a}FIC | Interpretation |
| S. aureus ATCC | mupirocin | 0.5 | 0.25 | 1 | Additive |
| 29213 | susceptible | | | | |
| S. aureus Oxford | mupirocin | 0.25 | 0.25 | 1 | Additive |
| | susceptible | | | | |
| S. aureus 31-1334 | low level mupirocin- | 0.5 | 32 | 0.75 | Additive |
| | resistant | | | | |
| S. aureus 14-354 | low level mupirocin- | 0.5 | 64 | 0.56 | Additive |
| | resistant | | | | |
| S. aureus 25-670 | high level | 0.5 | >128 | 2 | Indifferent |
| | mupirocin-resistant | | | | |

^{a}FIC = Fractional Inhibitory Concentration

Combination of mupirocin with the methionyl tRNA synthestase inhibitor (MRSi compound 2) showed additivity against mupirocin susceptible and low-level resistant strains of S. aureus. The combination was indifferent against the high-level resistant strain tested. Antagonism was not detected against the five strains tested in this study.

### EXAMPLE 18

### Study to Determine the Ability of an MRS Inhibitor (MRSI Compound 2) in Combination with Mupirocin to Select for Spontaneous Resistant Mutants of S. aureus.

Many antimicrobial agents have been shown to be capable of selecting for spontaneous resistant mutants. In recent years there has been increasing reports of both low and high-level mupirocin resistant staphylococci being isolated in the clinical setting. The objective of this study was to examine the ability of mupirocin and the MRS inhibitor alone and in combination to select for spontaneous resistant mutants of S. aureus.

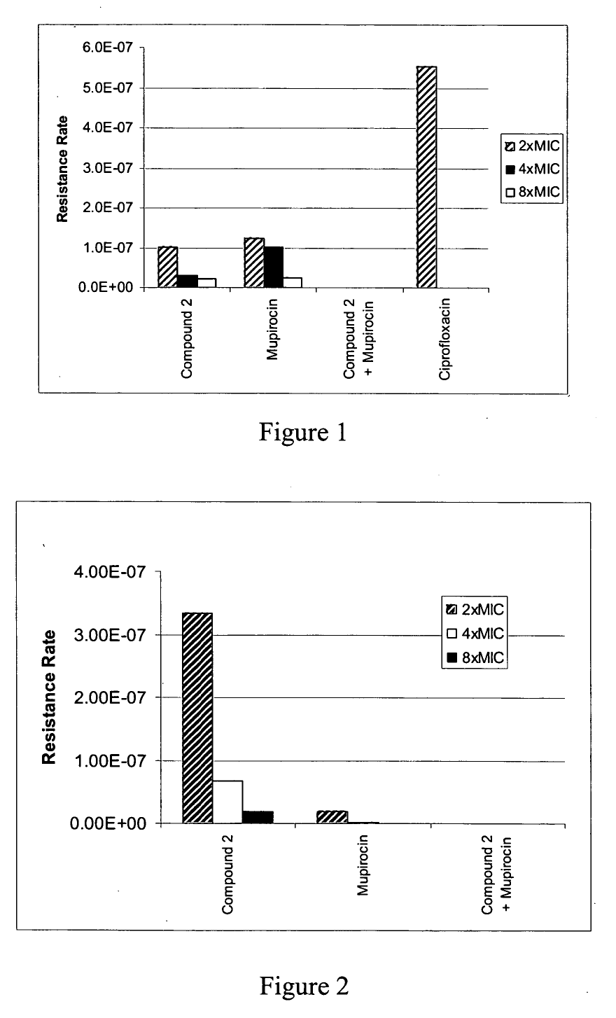

Approximately, 10⁹ bacteria were plated onto Mueller-Hinton agar supplemented with 10% horse blood and containing various concentrations of the test compounds alone and in combination. After 24 and 48 hours of incubation at 35° C., the bacterial colonies were counted and the frequencies of mutation were determined relative to the total viable count of organisms that were plated. Resistant clones were re-plated once on plates containing the same concentration of agent that was used for the selection. The results are summarized in Table 13 and FIGS. 1 and 2.

TABLE 13
Selection of tRNA synthetase inhibitor resistant mutants from S. aureus
ATCC 29213 and 31-1334

| | | Mutation Frequency (48 hours) | |
|---|---|---|---|
| | Concentration | S. aureus ATCC 29213 | S. aureus 31-1334 (low level |
| Selecting agent | (multiple of MIC) | (mupirocin susceptible) | mupirocin-resistant) |
| Mupirocin | 2 | 1.25 × 10⁻⁷ | 1.8 × 10⁻⁸ |
| | 4 | 1.01 × 10⁻⁷ | 2 × 10⁻⁹ |
| | 8 | 2.57 × 10⁻⁸ | <1 × 10⁻⁹ |
| MRSi compound 2 | 2 | 1.01 × 10⁻⁷ | 3.3 × 10⁻⁷ |
| | 4 | 3.14 × 10⁻⁸ | 6.8 × 10⁻⁸ |
| | 8 | 2.07 × 10⁻⁸ | 1.9 × 10⁻⁸ |
| Mupirocin + MRSi | 2 | <7 × 10⁻¹⁰ | <1 × 10⁻¹⁰ |
| compound 2 | 4 | <7 × 10⁻¹⁰ | <1 × 10⁻¹⁰ |
| | 8 | <7 × 10⁻¹⁰ | <1 × 10⁻¹⁰ |

Spontaneous resistant mutants were selected by plating S. aureus strains ATCC 29213 and 31-1334 onto medium containing mupirocin or MRSi compound 2 at 2, 4 and 8-fold MIC of each compound. No resistant colonies were detected on plates containing both compounds at 2, 4 and 8 their respective MICs. These results provide data to suggest that the combination of an MRS inhibitor (MRSi compound 2) with mupirocin (an IRS inhibitor) substantially reduces the propensity for the selection of resistant mutants from S. aureus.

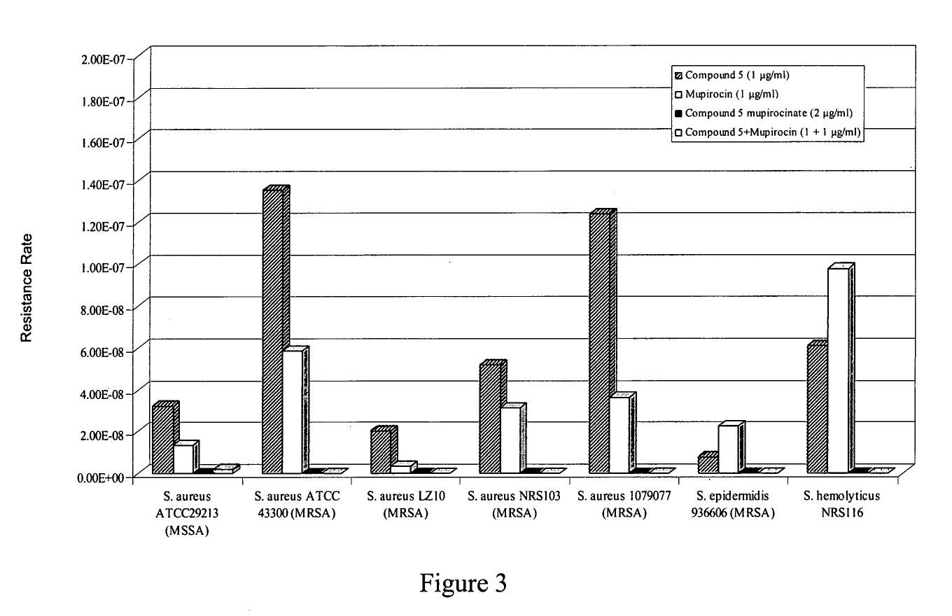

MRSi compound 5 (acetate salt, 1 μg/mL), mupirocin (1 μg/mL) and a 1:1 MRSi compound 5/mupirocin combination (each component at 1 μg/mL) were tested for their ability to select for spontaneous resistant mutants from seven different staphylococcal isolates. 10⁹ colony forming units (CFU) of each organism were incubated on media containing one of the single agents or the combination. The results in FIG. 3 show that both MRSi compound 5 and mupirocin had low propensity for the selection of spontaneous resistant mutants from staphylococci after incubation for 48 hours (resistance frequencies ranging from 3.3××10^{31 9} to 1.35×10⁻⁷). In contrast, no resistant colonies could be detected on media containing MRSi compound 5/mupirocin (1:1 combination) for any of the seven staphylococcal isolates tested after incubation for 48 hours.

### EXAMPLE 19

### Development of Resistance Following Serial Passage

MRSi compound 5 (acetate salt), mupirocin and MRSi compound 5/mupirocin 1:1 combination were tested for development of resistance following serial passage using 19 isolates, including S. aureus, coagulase-negative staphylococci and S. pyogenes strains. Serial passage in the presence of MRSi compound 5 alone resulted in isolates with elevated MICs after 20 passages (Table 14). The most resistant isolates that could be selected had MICs of 16 μg/mL and were observed with five of the organisms tested. In the case of the organisms passaged in the presence of the 1:1 MRSi compound 5/mupirocin combination, there was a lower propensity for the selection of isolates with elevated MICs. The most resistant mutant was obtained from a single isolate, S. aureus 1079101 (high-level mupirocin-resistant), that had an MIC of 8/8 μg/mL to the combination product following 20 passages.

TABLE 14
Susceptibility of Mutants Recovered from Serial Passage
Studies with REP258839 Alone and in 1:1
Combination with Mupirocin

| | MIC (μg/mL) | | | |
|---|---|---|---|---|
| | MRSi Compound 5 | | MRSi Compound | |
| | (acetate salt) | | 5/Mupirocin | |
| | | Final MIC | | Final MIC |
| | Initial | after 20 | Initial | after 20 |
| | MIC | passages | MIC | passages |
| Organism/Phenotype | (μg/mL) | (μg/mL) | (μg/mL) | (μg/mL) |
| S. aureus ATCC | 0.06 | 4 | 0.12/0.12 | 0.25/0.25 |
| 29213 | | | | |
| S. aureus ATCC | 0.06 | 1 | 0.12/0.12 | 0.5/0.5 |
| 43300 (ORSA) | | | | |
| S. aureus LZ10 | 0.03 | 0.5 | 0.12/0.12 | 0.5/0.5 |
| (ORSA) | | | | |
| S. aureus NRS103 | 0.12 | 1 | 0.06/0.06 | 0.5/0.5 |
| (ORSA) | | | | |
| S. aureus 1079077 | 0.25 | 16 | 0.25/0.25 | 0.5/0.5 |
| (ORSA) | | | | |
| S. aureus 31-1334 | 0.03 | 8 | 0.06/0.06 | 1/1 |
| (LL-MupR) | | | | |
| S. aureus NRS107 | 0.015 | 0.5 | 0.03/0.03 | 0.5/0.5 |
| (HL-MupR) | | | | |
| S. aureus LZ1 | 0.06 | 0.06 | 0.06/0.06 | 1/1 |
| (HL-MupR) | | | | |
| S. aureus LZ6 | 0.06 | 2 | 0.06/0.06 | 1/1 |
| (HL-MupR) | | | | |
| S. aureus 10-420 | 0.06 | 8 | 0.12/0.12 | 4/4 |
| (HL-MupR) | | | | |
| S. aureus 87-2797 | 0.03 | 16 | 0.06/0.06 | 0.5/0.5 |
| (HL-MupR) | | | | |
| S. aureus 25-670 | 0.06 | 8 | 0.12/0.12 | 4/4 |
| (HL-MupR) | | | | |
| S. aureus 1079101 | 0.06 | 16 | 0.12/0.12 | 8/8 |
| (HL-MupR) | | | | |
| S. epidermidis NRS8 | 0.06 | 0.12 | 0.12/0.12 | 1/1 |
| (LL-MupR) | | | | |
| S. epidermidis 936528 | 0.03 | 0.5 | 0.06/0.06 | 1/1 |
| (HL-MupR) | | | | |
| S. epidermidis 936606 | 0.06 | 16 | 0.12/0.12 | 0.12/0.12 |
| (Oxa-R) | | | | |
| S. hemolyticus NRS116 | 0.12 | 16 | 0.12/0.12 | 0.25/0.25 |
| S. pyogenes ATCC | 0.12 | 1 | 0.12/0.12 | 0.25/0.25 |
| 19615 | | | | |
| S. pyogenes MB000143 | 0.12 | 1 | 0.12/0.12 | 0.25/0.25 |
| (Ery-R) | | | | |

### EXAMPLE 20

### Susceptibility of MRS-Resistant Mutants of S. aureus to Mupirocin and 1:1 MRSI Compound 5/Mupirocin Combination

MRS-resistant mutants generated in vitro in either serial passage or spontaneous resistance development studies were evaluated for susceptibility to mupirocin alone and in a 1:1 combination with MRSi compound 5. All mutants were characterized to identify key mutations in metS (Table 15). All the MRS-resistant mutants retained susceptibility to mupirocin and 1:1 MRSi compound 5/mupirocin with MICs ranging from 0.12 to 1 μg/mL and 0.06/0.06 to 1/1 μg/mL respectively indicating little or no cross resistance between the two targets.

TABLE 15
Susceptibility of MRS-resistant Mutants of S. aureus to
Mupirocin Alone and in 1:1 Combination with MRSi Compound 5

| | | MIC (μg/ml) | | |
|---|---|---|---|---|
| | | MRSi | | MRSi |
| | metS | Cmpd 5 | | Cmpd |
| Organism/mutant | mutation(s) | (acetate salt) | Mupirocin | 5/mupirocin |
| S. aureus ATCC | wild-type | 0.06 | 0.12 | 0.06/0.06 |
| 29213 | | | | |
| S. aureus ATCC | wild-type | 0.06 | 0.12 | 0.12/0.12 |
| 43300 | | | | |
| S. aureus SP-1A2 | A247E | 4 | 0.25 | 0.25/0.25 |
| S. aureus SP-1B5 | I57N | 8 | 0.12 | 0.25/0.25 |
| S. aureus SP-9B5 | I57N, V296F | 4 | 0.5 | 1/1 |
| S. aureus SP-2B5 | I57N, R100S | 16 | 0.12 | 0.25/0.25 |
| S. aureus SP-2C4 | L213W | 4 | 0.12 | 0.25/0.25 |
| S. aureus SP-2D4 | I57N | 16 | 0.25 | 0.25/0.25 |
| S. aureus SP-21A | A77V | 4 | 1 | 1/1 |
| S. aureus SR1 | I57N | 4 | 0.12 | 0.25/0.25 |

### EXAMPLE 21

### Growth Curve Analysis of MRS-Resistant Mutants

MRS-resistant mutants, S. aureus SP-1A2 and S. aureus SP-1B5 were evaluated in a growth curve study along with the parent wild type strain (S. aureus ATCC 29213). Growth of all three strains was determined by monitoring optical density (600 nm) over eight hours.

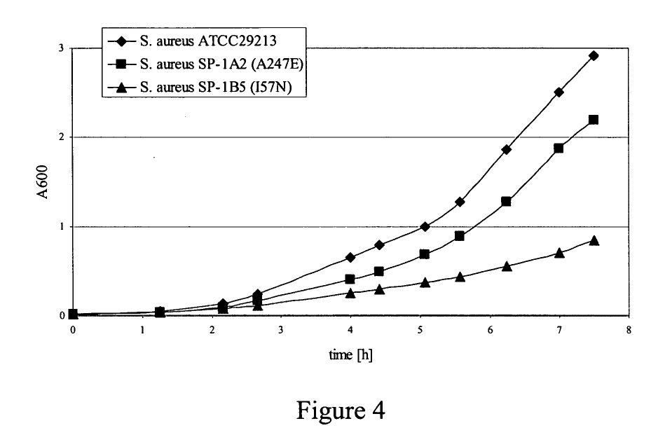

The results in FIG. 4 show that both the resistant mutants have slower rates of growth when compared with the wild type parent strain. It is possible that the A247E and 157N mutations appear to be responsible for low-level resistance to MRSi compound 5 and may also be associated with a fitness burden cost to the cell.

### EXAMPLE 22

### Mode of Action Confirmation Studies

To confirm its mode of action, MRSi compound 5 was tested against a strain of S. aureus in which the metRS gene was placed under the control of a xyl/tet promoter on a S. aureus compatible plasmid. The strain expresses high levels of MRS upon the addition of 0.01 μg/mL of anyhdrotetracycline. The results in Table 16 show that over-expression of MRS leads to an 8-fold increase in MIC for MRSi compound 5 but not for mupirocin or the other control compounds tested.

TABLE 16
Effect of MRS over-production in S. aureus on the
antibacterial activity of MRS Inhibitor Compound 5

S. aureus RN4220
(pYH4-MRS)

| | −aTC*) | +aTC |
|---|---|---|
| Compound | MIC [μg/ml] | MIC [μg/ml] |
| MRSi Cmpd 5 | 0.12 | 1 |
| Mupirocin | 0.06 | 0.06 |
| Novobiocin | 0.25 | 0.25 |
| Vancomycin | 0.5 | 0.5 |

*)aTC, anhydrotetracycline

What is claimed is:

1. A composition comprising an aminoacyl tRNA synthetase inhibitor or a pharmaceutically acceptable salt thereof, and an additional antibacterial agent.

2. A composition comprising a combination of two or more aminoacyl tRNA synthetase inhibitors, or the pharmaceutically acceptable salts thereof.

3. A composition comprising a methionyl tRNA synthetase inhibitor or a pharmaceutically acceptable salt thereof and an antibacterial agent.

4. The composition of claim 3 wherein the antibacterial agent is an aminoacyl tRNA synthetase inhibitor or a pharmaceutically acceptable salt thereof.

5. The composition of claim 4 wherein the aminoacyl tRNA synthetase inhibitor is an isoleucyl tRNA synthetase inhibitor.

6. The composition of claim 5 wherein the isoleucyl tRNA synthetase inhibitor is mupirocin or a pharmaceutically acceptable salt or ester thereof.

7. The composition of claim 3 wherein the methionyl tRNA synthetase inhibitor is selected from the group consisting of: 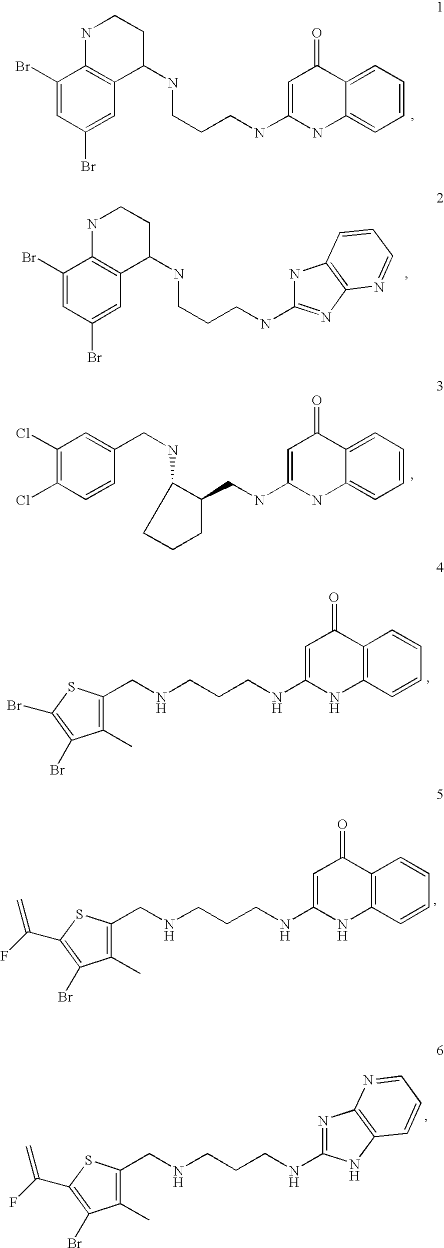 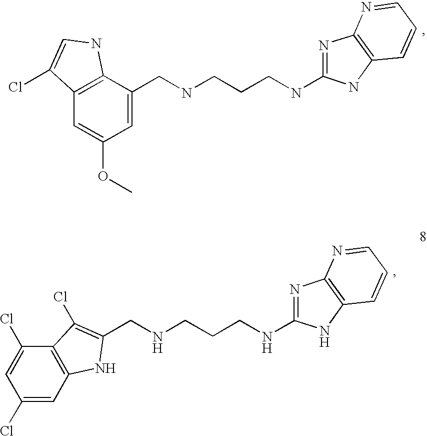
and a pharmaceutically acceptable salt of any of the foregoing compounds.

8. The composition of claim 7 wherein the methionyl tRNA synthetase inhibitor is 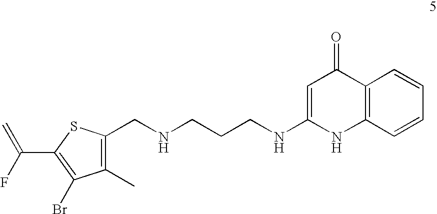
or a pharmaceutically acceptable salt thereof and the antibacterial agent is mupirocin or a pharmaceutically acceptable salt or ester thereof.

9. A pharmaceutical composition for topical application to humans or domestic mammals comprising mupirocin or a pharmaceutically acceptable salt or ester thereof, and at least one additional tRNA synthetase inhibitor or a pharmaceutically acceptable salt thereof.

10. A composition comprising a salt of a methionyl tRNA synthetase inhibitor wherein the salt is selected from the group consisting of the Mupirocinate salt and the Fusidate salt.

11. The composition claim 10, wherein the methionyl tRNA synthetase inhibitor is selected from the group consisting of:

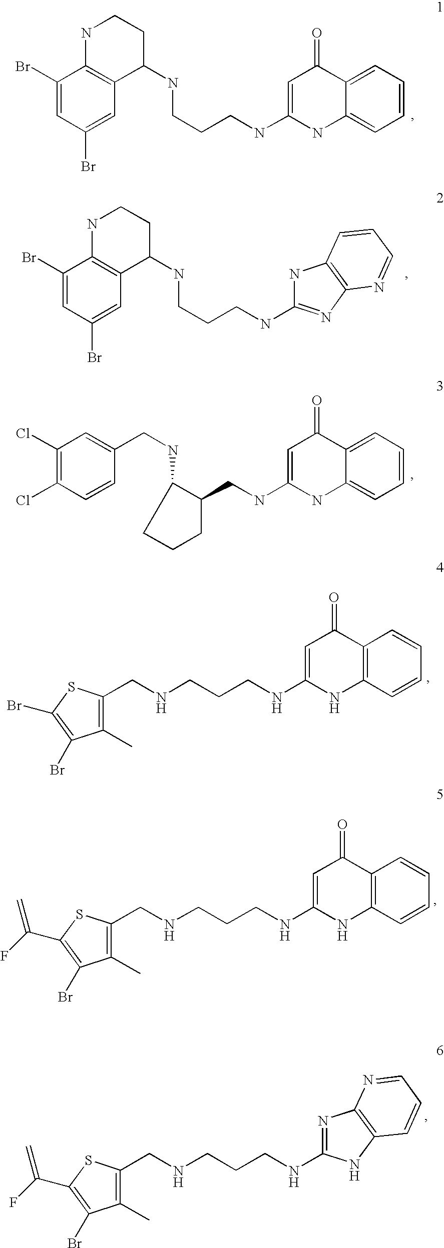

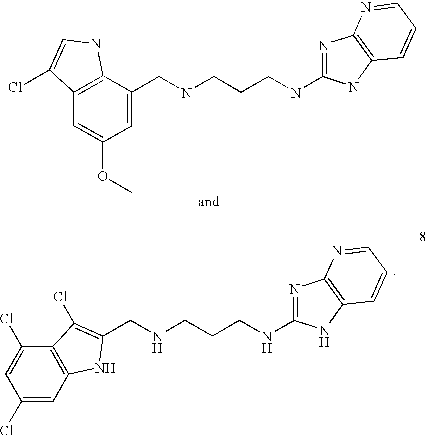

12. The composition of claim 11 comprising N-(4,5-Dibromo-3-methylthiophen-2-ylmethyl)-N′-(1H-quinolin-4-one)propane-1,3-diamine Mupirocinate.

13. The composition of claim 11 composition comprising N-(4-bromo-5-(1-fluorovinyl)-3-methylthiophen-2-ylmethyl)-N′-(1H-quinolin-4-one)propane-1,3-diamine Mupirocinate.

14. The composition of claim 11 composition comprising N-(3-Chloro-5-methoxy-1H-indol-2-ylmethyl)-N′-(1H-imidazo[4,5-b]pyridin-2-yl)-propane-1,3-diamine Mupirocinate.

15. The composition of claim 11 comprising N-(1H-imidazo[4,5-b]pyridin-2-yl)-N′-(3,4,6-trichloro-1H-indol-2-ylmethyl)-propane-1,3-diamine Mupirocinate.

16. The composition of claim 11 comprising N-(1H-imidazo[4,5-b]pyridin-2-yl)-N′-(3,4,6-trichloro-1H-indol-2-ylmethyl)-propane-1,3-diamine Fusidinate.

17. A method of treating a bacterial infection, comprising administering the pharmaceutical composition of claim 2 to a host having a bacterial infection.

18. The method of claim 17, wherein the bacterial infection is an infection of a an enterococcus.

19. The method of claim 18, wherein the enterococcus is selected from the group consisting of E. faecalis and E. faecium.

20. The method of claim 17, wherein the enterococcus is a vancomycin-resistant strain.

21. The method of claim 17, wherein the bacterial infection is an infection of a bacterium selected from the group consisting of S. aureus, S. pyogenes, S. epidermidis, and S. hemolyticus.

22. The method of claim 21, wherein the S. aureus is selected from the group consisting of vancomycin-intermediate S. aureus, low-level mupirocin-reistant S. aureus and high-level mupirocin-reistant S. aureus.

23. The method of claim 17, wherein the bacterial infection is an infection of a bacterium selected from the group consisting of S. aureus, S. pyogenes, S. epidermidis, and S. hemolyticus.

24. The method of claim 21, wherein the S. aureus is selected from the group consisting of vancomycin-intermediate S. aureus, low-level mupirocin-reistant S. aureus and high-level mupirocin-reistant S. aureus.
